(19) United States
(12) Patent Application Publication
(10) Pub. No.: US 2021/0154170 A1
(43) Pub. Date: May 27, 2021

(54) METHODS OF TREATING NEUROPATHIC PAIN

(71) Applicant: Biogen MA Inc., Cambridge, MA (US)

(72) Inventors: Yuan Zhao, Cambridge, MA (US); Himanshu Naik, Cambridge, MA (US)

(21) Appl. No.: 17/047,570

(22) PCT Filed: Apr. 12, 2019
(86) PCT No.: PCT/US19/27158

§ 371 Date: Oct. 14, 2020

Related U.S. Application Data
(60) Provisional application No. 62/658,347, filed on Apr. 16, 2018.

(51) Int. Cl. A61K 31/401; A61P 25/02

(52) U.S. Cl. CPC A61K 31/401; A61P 25/02

(57) ABSTRACT

The present disclosure provides methods of treatment using (5R)-5-(4-{[(2-fluorophenyl)methyl]oxy}phenyl)-L-prolinamide and pharmaceutically salts thereof.

### RELATED APPLICATIONS

This application claims the benefit of priority to U.S. Provisional Application No. 62/658,347, filed Apr. 16, 2018, the contents of which are hereby incorporated herein by reference in their entirety.

### BACKGROUND

(2S,5R)-5-(4-((2-fluorobenzyl)oxy)phenyl)pyrrolidine-2-carboxamide, herein referred to as the compound of formula (I):

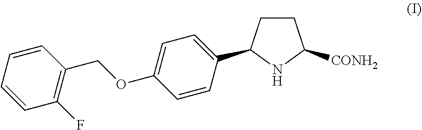 [SMILES: NC(=O)[C@@H]1CC[C@H](c2ccc(OCc3ccccc3F)cc2)N1]

is described in U.S. Pat. No. 7,655,693 as having utility in the treatment of diseases and conditions mediated by state-dependent modulation of Nav1.7 and/or other voltage-gated sodium channel subtypes.

However, there is a need for the development of improved dosage regimens to optimize the treatment of patients suffering from disorders such as trigeminal neuralgia and to minimize their debilitating symptoms.

### SUMMARY

Provided herein are methods of treating a disease or condition mediated by modulation of Nav1.7 by administering (5R)-5-(4-{[(2-fluorophenyl)methyl]oxy}phenyl)-L-prolinamide, or a pharmaceutically acceptable salt thereof, to a subject not receiving treatment with a UGT inhibitor. In certain embodiments, the disease or condition is associated with a defect or dysfunction of Nav1.7.

Also provided herein are methods of treating a disease or condition mediated by modulation of Nav1.7 by administering (5R)-5-(4-{[(2-fluorophenyl)methyl]oxy}phenyl)-L-prolinamide, or a pharmaceutically acceptable salt thereof, to a subject, wherein the subject is receiving treatment with a UGT inhibitor. In certain embodiments, the disease or condition is associated with a defect or dysfunction of Nav1.7.

In some embodiments, (5R)-5-(4-{[(2-fluorophenyl)methyl]oxy}phenyl)-L-prolinamide, or a pharmaceutically acceptable salt thereof, is administered one time per day (OID). In other embodiments, (5R)-5-(4-{[(2-fluorophenyl)methyl]oxy}phenyl)-L-prolinamide, or a pharmaceutically acceptable salt thereof, is administered two times per day (BID). In yet other embodiments, (5R)-5-(4-{[(2-fluorophenyl)methyl]oxy}phenyl)-L-prolinamide, or a pharmaceutically acceptable salt thereof, is administered three times per day (TID).

In some embodiments, (5R)-5-(4-{[(2-fluorophenyl)methyl]oxy}phenyl)-L-prolinamide, or a pharmaceutically acceptable salt thereof, is administered at a dosage of about 50 mg to about 400 mg.

In some embodiments, the disease or condition is pain. In preferred embodiments, the pain is neuropathic pain, such as diabetic neuropathy; sciatica; non-specific lower back pain; painful lumbosacral radiculopathy; multiple sclerosis pain; fibromyalgia; HIV-related neuropathy; post-herpetic neuralgia; trigeminal neuralgia; or pain resulting from physical trauma, amputation, cancer, toxins, or a chronic inflammatory condition.

### BRIEF DESCRIPTION OF THE DRAWINGS

FIG. 1 shows PK modelling plots. For all doses, the C Trough is higher than efficacious doses in animal model of inflammation. FCA5 corresponds to an oral dose of 5 mg/kg, which fully reversed the hyperalgesia in the Freud Complete Adjuvant induced inflammation model. FCA1, an oral dose of 1 mg/kg was the minimal effective dose in this model. TGN (trigeminal neuralgia), PLSR (painful lumbosacral radiculopathy).

FIG. 2 shows the design of 300/400 mg BID Dosage study.

FIG. 3 shows the change in Outpatient 24 h SBP (A) and DBP (B) from Baseline to Day 36.

FIG. 4 shows the proportion of Observations with Changes in Outpatient 24 h SBP (A) or DBP (B) on Day 36 Compared to Baseline.

FIG. 5 shows the change in Inpatient 12 h SBP (A) and DBP (B) from Baseline to Day 35.

FIG. 6 shows the arithmetic mean (+/−SD) plasma concentration profiles for BIIB074 (ng/mL) following treatment with BIIB074 alone or in combination with valproic acid. Exposure of BIIB074 (AUC) increased after administration of BIIB074 with valproic acid compared to administration of BIIB074 alone. There was no change in Cₘₐₓ. Elimination was prolonged.

FIG. 7 shows the arithmetic mean (+/−SD) plasma concentration profiles for the UGT-derived BIIB074 metabolite M13 (ng/mL) following treatment with BIIB074 alone or in combination with valproic acid. Exposure of the UGT-derived metabolite M13 (AUC and Cₘₐₓ) was reduced after administration of BIIB074 with valproic acid compared to BIIB074 alone.

FIG. 8 shows the arithmetic mean (+/−SD) plasma concentration profiles for M14 (ng/mL) following treatment with BIIB074 alone or in combination with valproic acid.

Exposure of M14 (AUC and Cₘₐₓ) increased after administration of BIIB074 with valproic acid compared to BIIB074 alone.

FIG. 9 shows the arithmetic mean (+/−SD) plasma concentration profiles for M16 (ng/mL) following treatment with BIIB074 alone or in combination with valproic acid. Exposure of M16 (AUC and Cₘₐₓ) increased after administration of BIIB074 with valproic acid compared to BIIB074 alone.

### DETAILED DESCRIPTION

According to some embodiments, provided herein are methods of treating a disease or condition mediated by modulation of Nav1.7 and/or another voltage-gated sodium channel subtype in a patient in need thereof by administering (5R)-5-(4-{[(2-fluorophenyl)methyl]oxy}phenyl)-L-prolinamide, or a pharmaceutically acceptable salt thereof. In certain embodiments, the disease or condition is associated with a defect or dysfunction of Nav1.7.

In some embodiments, (5R)-5-(4-{[(2-fluorophenyl)methyl]oxy}phenyl)-L-prolinamide, or a pharmaceutically acceptable salt thereof, is administered one time per day (OID). In other embodiments, (5R)-5-(4-{[(2-fluorophenyl)methyl]oxy}phenyl)-L-prolinamide, or a pharmaceutically acceptable salt thereof, is administered two times per day (BID). In yet other embodiments, (5R)-5-(4-{[(2-fluorophenyl)methyl]oxy}phenyl)-L-prolinamide, or a pharmaceutically acceptable salt thereof, is administered three times per day (TID).

In some embodiments, (5R)-5-(4-{[(2-fluorophenyl)methyl]oxy}phenyl)-L-prolinamide, or a pharmaceutically acceptable salt thereof, is administered at a dosage of about 50 mg to about 400 mg. In some such embodiments, the dosage may be about 50 mg to about 400 mg, about 75 mg to about 400 mg, about 100 mg to about 400 mg, about 125 mg to about 400 mg, about 150 mg to about 400 mg, about 175 mg to about 400 mg, about 200 mg to about 400 mg, about 225 mg to about 400 mg, about 250 mg to about 400 mg, about 275 mg to about 400 mg, about 300 mg to about 400 mg, about 325 mg to about 400 mg, about 350 mg to about 400 mg, about 375 mg to about 400 mg, about 50 mg to about 350 mg, about 50 mg to about 325 mg, about 50 mg to about 300 mg, about 50 mg to about 275 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, or about 50 mg to about 75 mg. In certain embodiments, the dosage may be about 50 mg, about 75 mg about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg. In certain embodiments, the dosage is about 50 mg, about 75 mg, about 100 mg, about 150 mg, about 200 mg, or about 350 mg. In certain other embodiments, the dosage is about 50 mg, 75 mg, 100 mg, 150 mg, or 250 mg. In certain embodiments, the doses listed above are administered one time per day (OID). In other embodiments, the doses listed above are administered two times per day (BID). In other embodiments, the doses listed above are administered three times per day (TID).

In some embodiments, (5R)-5-(4-{[(2-fluorophenyl)methyl]oxy}phenyl)-L-prolinamide is administered at a dosage of about 200 mg two times per day (BID), or at a dosage of about 150 mg or about 250 mg three times per day (TID). In certain such embodiments, the dosage of about 150 mg is administered only to a subject identified as a responder to treatment with (5R)-5-(4-{[(2-fluorophenyl)methyl]oxy}phenyl)-L-prolinamide. In certain embodiments, the (5R)-5-(4-{[(2-fluorophenyl)methyl]oxy}phenyl)-L-prolinamide is provided as a hydrochloride salt.

In some embodiments, (5R)-5-(4-{[(2-fluorophenyl)methyl]oxy}phenyl)-L-prolinamide, or a pharmaceutically acceptable salt thereof, is administered at a dosage of about 200 mg two times per day (BID), such as for treating painful lumbosacral radiculopathy (PLSR) to a subject. In certain such embodiments, the dosage of about 200 mg BID is administered only to a subject identified as a responder to treatment with (5R)-5-(4-{[(2-fluorophenyl)methyl]oxy}phenyl)-L-prolinamide. In certain embodiments, the (5R)-5-(4-{[(2-fluorophenyl)methyl]oxy}phenyl)-L-prolinamide is provided as a hydrochloride salt.

In some embodiments, (5R)-5-(4-{[(2-fluorophenyl)methyl]oxy}phenyl)-L-prolinamide, or a pharmaceutically acceptable salt thereof, is administered at a dosage of about 150 mg three times per day (TID). In certain such embodiments, the dosage of about 150 mg is administered only to subjects identified as a responders to treatment with (5R)-5-(4-{[(2-fluorophenyl)methyl]oxy}phenyl)-L-prolinamide. In certain embodiments, the (5R)-5-(4-{[(2-fluorophenyl)methyl]oxy}phenyl)-L-prolinamide is provided a hydrochloride salt.

In some embodiments, (5R)-5-(4-{[(2-fluorophenyl)methyl]oxy}phenyl)-L-prolinamide, or a pharmaceutically acceptable salt thereof, is administered at a dosage of about 250 mg three times per day (TID), such as for treating trigeminal neuralgia (TN) in a subject in need thereof. In certain embodiments, the (5R)-5-(4-{[(2-fluorophenyl)methyl]oxy}phenyl)-L-prolinamide is provided as a hydrochloride salt.

In some embodiments, a dosage of about 250 mg is administered to a subject not previously treated with (5R)-5-(4-{[(2-fluorophenyl)methyl]oxy}phenyl)-L-prolinamide. In alternative embodiments, a dosage of about 250 mg is administered to a subject previously treated with a dosage of about 150 mg of (5R)-5-(4-{[(2-fluorophenyl)methyl]oxy}phenyl)-L-prolinamide, or a pharmaceutically acceptable salt thereof, and wherein the subject has been identified as a non-responder to treatment with the dosage of 150 mg of (5R)-5-(4-{[(2-fluorophenyl)methyl]oxy}phenyl)-L-prolinamide. In certain embodiments, the (5R)-5-(4-{[(2-fluorophenyl)methyl]oxy}phenyl)-L-prolinamide is provided as a hydrochloride salt.

Also provided herein are methods of treating a disease or condition mediated by modulation of Nav1.7 and/or another voltage-gated sodium channel subtype in a patient in need thereof which comprises administering (5R)-5-(4-{[(2-fluorophenyl)methyl]oxy}phenyl)-L-prolinamide, or a pharmaceutically acceptable salt thereof, to a subject at a dosage of about 300 mg to about 400 mg two times per day (BID). In some such embodiments, the dosage regimen may not result in a clinically relevant change in systolic blood pressure (SBP) and diastolic blood pressure (DBP) following dosage for up to 36 days (see the results of the study shown in Example 4).

In some embodiments, a dosage of about 300 mg BID is administered to a female patient. In further embodiments, the dosage of about 300 mg BID is administered following a dosage of about 400 mg BID for an initial period of time (such as, for example, approximately 1 week).

In other embodiments, a dosage of about 400 mg BID is administered to a male patient.

As used herein, the phrase “is administered to a subject a dosage of” is meant to indicate that the free base form of (5R)-5-(4-{[(2-fluorophenyl)methyl]oxy}phenyl)-L-prolinamide is delivered in the recited amount. For example, if the free base form of (5R)-5-(4-{[(2-fluorophenyl)methyl]oxy}phenyl)-L-prolinamide is “administered at a dosage of about 150 mg” in tablet form, the tablet would contain about 150 mg of the free base of (5R)-5-(4-{[(2-fluorophenyl)methyl]oxy}phenyl)-L-prolinamide. Furthermore, if the free base form of (5R)-5-(4-{[(2-fluorophenyl)methyl]oxy}phenyl)-L-prolinamide is “administered at a dosage of about 250 mg” in tablet form, the tablet would contain about 250 mg of the free base of (5R)-5-(4-{[(2-fluorophenyl)methyl]oxy}phenyl)-L-prolinamide. Furthermore, if the free base form of (5R)-5-(4-{[(2-fluorophenyl)methyl]oxy}phenyl)-L-prolinamide is “administered at a dosage of about 300 mg” in tablet form, the tablet would contain about 300 mg of the free base of (5R)-5-(4-{[(2-fluorophenyl)methyl]oxy}phenyl)-L-prolinamide. Furthermore, if the free base form of (5R)-5-(4-{[(2-fluorophenyl)methyl]oxy}phenyl)-L-prolinamide is “administered at a dosage of about 400 mg” in tablet form, the tablet would contain about 400 mg of the free base of (5R)-5-(4-{[(2-fluorophenyl)methyl]oxy}phenyl)-L-prolinamide. If (5R)-5-(4-{[(2-fluorophenyl)methyl]oxy}phenyl)-L-prolinamide in the form of a hydrochloride salt is “administered at a dosage of about 150 mg” in tablet form, the tablet would contain about 167 mg of the hydrochloride salt of (5R)-5-(4-{[(2-fluorophenyl)methyl]oxy}phenyl)-L-prolinamide. Furthermore, if (5R)-5-(4-{[(2-fluorophenyl)methyl]oxy}phenyl)-L-prolinamide in the form of a hydrochloride salt is “administered at a dosage of about 200 mg” in tablet form, the tablet would contain about 223 mg of the hydrochloride salt of (5R)-5-(4-{[(2-fluorophenyl)methyl]oxy}phenyl)-L-prolinamide. Furthermore, if (5R)-5-(4-{[(2-fluorophenyl)methyl]oxy}phenyl)-L-prolinamide in the form of a hydrochloride salt is “administered at a dosage of about 250 mg” in tablet form, the tablet would contain about 279 mg of the hydrochloride salt of (5R)-5-(4-{[(2-fluorophenyl)methyl]oxy}phenyl)-L-prolinamide. Furthermore, if (5R)-5-(4-{[(2-fluorophenyl)methyl]oxy}phenyl)-L-prolinamide in the form of a hydrochloride salt is “administered at a dosage of about 300 mg” in tablet form, the tablet would contain about 334 mg of the hydrochloride salt of (5R)-5-(4-{[(2-fluorophenyl)methyl]oxy}phenyl)-L-prolinamide. Furthermore, if (5R)-5-(4-{[(2-fluorophenyl)methyl]oxy}phenyl)-L-prolinamide in the form of a hydrochloride salt is “administered at a dosage of about 400 mg” in tablet form, the tablet would contain about 446 mg of the hydrochloride salt of (5R)-5-(4-{[(2-fluorophenyl)methyl]oxy}phenyl)-L-prolinamide.

Also provided herein are methods of treating a disease or condition mediated by modulation of Nav1.7 comprising administering (5R)-5-(4-{[(2-fluorophenyl)methyl]oxy}phenyl)-L-prolinamide, or a pharmaceutically acceptable salt thereof, to a subject who is not receiving treatment with a UGT inhibitor. In certain embodiments, the disease or condition is associated with a defect or dysfunction of Nav1.7.

In some embodiments, the method further comprises determining whether the subject is receiving treatment with a UGT inhibitor. If the subject is receiving treatment with a UGT inhibitor, the subject may be instructed to discontinue treatment with the UGT inhibitor prior to commencing treatment with (5R)-5-(4-{[(2-fluorophenyl)methyl]oxy}phenyl)-L-prolinamide, or a pharmaceutically acceptable salt thereof. If, however, the subject is not receiving treatment with a UGT inhibitor, the subject may be instructed not to commence treatment with a UGT inhibitor while receiving treatment with (5R)-5-(4-{[(2-fluorophenyl)methyl]oxy}phenyl)-L-prolinamide, or a pharmaceutically acceptable salt thereof.

In some embodiments, a subject that has been receiving treatment with a UGT inhibitor is instructed to stop using the UGT inhibitor before beginning administration of (5R)-5-(4-{[(2-fluorophenyl)methyl]oxy}phenyl)-L-prolinamide, or a pharmaceutically acceptable salt thereof. For example, the subject may be instructed to stop using the UGT inhibitor at least three weeks before beginning administration of (5R)-5-(4-{[(2-fluorophenyl)methyl]oxy}phenyl)-L-prolinamide, or a pharmaceutically acceptable salt thereof. Similarly, the subject may be instructed to stop using the UGT inhibitor at least two weeks before beginning administration of (5R)-5-(4-{[(2-fluorophenyl)methyl]oxy}phenyl)-L-prolinamide, or a pharmaceutically acceptable salt thereof. Alternatively, the subject may be instructed to stop using the UGT inhibitor at least one week before beginning administration of (5R)-5-(4-{[(2-fluorophenyl)methyl]oxy}phenyl)-L-prolinamide, or a pharmaceutically acceptable salt thereof.

Also provided herein are methods of treating a disease or condition mediated by modulation of Nav1.7 by administering (5R)-5-(4-{[(2-fluorophenyl)methyl]oxy}phenyl)-L-prolinamide, or a pharmaceutically acceptable salt thereof, to a subject receiving treatment with a UGT inhibitor.

In some such embodiments, the subject's dosage of (5R)-5-(4-{[(2-fluorophenyl)methyl]oxy}phenyl)-L-prolinamide, or a pharmaceutically acceptable salt thereof, is lowered at least 30% relative to what it would have been had the subject not been using a UGT inhibitor. Alternatively, the subject's dosage of (5R)-5-(4-{[(2-fluorophenyl)methyl]oxy}phenyl)-L-prolinamide, or a pharmaceutically acceptable salt thereof, may be lowered at least 50% relative to what it would have been had the subject not been using a UGT inhibitor. In certain embodiments, the subject's dosage of (5R)-5-(4-{[(2-fluorophenyl)methyl]oxy}phenyl)-L-prolinamide, or a pharmaceutically acceptable salt thereof, may be a dosage of 250 mg TID.

In some embodiments, the dosage of (5R)-5-(4-{[(2-fluorophenyl)methyl]oxy}phenyl)-L-prolinamide administered to a subject not receiving treatment with a UGT inhibitor (for instance, as used to determine the dosage of (5R)-5-(4-{[(2-fluorophenyl)methyl]oxy}phenyl)-L-prolinamide to be administered to a subject who is receiving treatment with a UGT inhibitor) is that dosage which would be prescribed by a physician in accordance with prescribing guidelines (such as those found on an FDA label). In certain embodiments, the dosage of a subject not receiving treatment with a UGT inhibitor is one of the dosages described elsewhere herein. For example, in certain embodiments, the dosage of a subject not receiving treatment with a UGT inhibitor is about 150 mg to about 400 mg, e.g., about 200 mg to about 400 mg, about 250 mg to about 400 mg, about 300 mg to about 400 mg, about 350 mg to about 400 mg, about 150 mg to about 350 mg, about 150 mg to about 300 mg, about 150 mg to about 250 mg, or about 150 mg to about 200 mg. In particular embodiments, the dosage of a subject not receiving treatment with a UGT inhibitor is about 150 mg, about 200 mg, about 250 mg, about 300 mg, about 350 mg, or about 400 mg. In preferred embodiments, the dosage of a subject not receiving treatment with a UGT inhibitor is about 150 mg, about 200 mg, about 250 mg, about 300 mg, or about 400 mg.

In certain embodiments, the methods of treating a disease or condition mediated by modulation of Nav1.7 (such as painful lumbosacral radiculopathy) comprise administering (5R)-5-(4-{[(2-fluorophenyl)methyl]oxy}phenyl)-L-prolinamide, or a pharmaceutically acceptable salt thereof, at a dosage of about 50 mg to about 350 mg BID to a subject receiving treatment with a UGT inhibitor. In certain embodiments, the dosage is about 50 mg BID, about 75 mg BID, about 100 mg BID, about 150 mg BID, about 200 mg BID, or about 350 mg BID.

In certain embodiments, the methods of treating a disease or condition mediated by modulation of Nav1.7 (such as trigeminal neuralgia) comprise administering (5R)-5-(4-{[(2-fluorophenyl)methyl]oxy}phenyl)-L-prolinamide, or a pharmaceutically acceptable salt thereof, at a dosage of about 50 mg to about 250 mg TID to a subject receiving treatment with a UGT inhibitor. In certain embodiments, the dosage is about 50 mg TID, about 75 mg TID, about 100 mg TID, about 150 mg TID, or about 250 mg TID.

In certain embodiments for treating a subject receiving treatment with a UGT inhibitor, the method comprises instructing the subject to lower the dosage of the UGT inhibitor before beginning administration of (5R)-5-(4-{[(2-fluorophenyl)methyl]oxy}phenyl)-L-prolinamide, or a pharmaceutically acceptable salt thereof. For example, the subject may be instructed to lower the dosage of the UGT inhibitor at least three weeks before beginning administration of (5R)-5-(4-{[(2-fluorophenyl)methyl]oxy}phenyl)-L-prolinamide, or a pharmaceutically acceptable salt thereof. Similarly, the subject may be instructed to lower the dosage of the UGT inhibitor at least two weeks before beginning administration of (5R)-5-(4-{[(2-fluorophenyl)methyl]oxy}phenyl)-L-prolinamide, or a pharmaceutically acceptable salt thereof. Alternatively, the subject may be instructed to lower the dosage of the UGT inhibitor at least one week before beginning administration of (5R)-5-(4-{[(2-fluorophenyl)methyl]oxy}phenyl)-L-prolinamide, or a pharmaceutically acceptable salt thereof. In certain embodiments, the method comprises instructing the subject to discontinue treatment with the UGT inhibitor.

Examples of suitable UGT inhibitors include but are not limited to canagliflozin, dapagliflozin, mefenamic acid, probenecid, diclofenac, quinidine, fluconazole, and valproic acid. In preferred embodiments, the UGT inhibitor is valproic acid.

In certain embodiments, said disease or condition is pain. For example, the disease or condition may be chronic inflammatory pain (e.g., pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

In some embodiments, the pain is neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. In certain embodiments, the neuropathic pain is selected from: diabetic neuropathy; sciatica; non-specific lower back pain; painful lumbosacral radiculopathy; multiple sclerosis pain; fibromyalgia; HIV-related neuropathy; post-herpetic neuralgia; trigeminal neuralgia; erythromelalgia; small fibre neuropathy; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, neuropathic pain includes pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

In preferred embodiments, the neuropathic pain is selected from trigeminal neuralgia, painful lumbosacral radiculopathy, erythromelalgia, and small fibre neuropathy. In the most preferred embodiments, the neuropathic pain is trigeminal neuralgia or painful lumbosacral radiculopathy.

In some embodiments, the disease or condition is an inflammatory disorder, such as a skin condition (e.g., sunburn, burns, eczema, dermatitis, psoriasis); ophthalmic disease; lung disorder (e.g., asthma, bronchitis, emphysema, allergic rhinitis, non-allergic rhinitis, cough, respiratory distress syndrome, pigeon fancier's disease, farmer's lung, chronic obstructive pulmonary disease, (COPD); gastrointestinal tract disorder (e.g., Crohn's disease, ulcerative colitis, coeliac disease, regional ileitis, irritable bowel syndrome, inflammatory bowel disease, gastroesophageal reflux disease); or other condition with an inflammatory component such as migraine, multiple sclerosis, myocardial ischemia.

Without wishing to be bound by theory, other diseases or conditions that may be mediated by modulation of Nav1.7 and/or another voltage-gated sodium channel subtype are selected from the list consisting of [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90);

ii) Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9);

iii) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00);

iv) Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide;

v) Enhancement of cognition including the treatment of cognition impairment in other diseases such as schizophrenia, bipolar disorder, depression, other psychiatric disorders and psychotic conditions associated with cognitive impairment, e.g., Alzheimer's disease;

vi) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome;

vii) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50);

viii) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism);

ix) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23);

x) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9);

xi) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9); and

xii) Impulse control disorder” including: Intermittent Explosive Disorder (312.34), Kleptomania (312.32), Pathological Gambling (312.31), Pyromania (312.33), Trichotillomania (312.39), Impulse-Control Disorders Not Otherwise Specified (312.3), Binge Eating, Compulsive Buying, Compulsive Sexual Behaviour and Compulsive Hoarding.

In some embodiments, the diseases or conditions that may be mediated by modulation of Nav1.7 and/or other voltage-gated sodium channels are depression or mood disorders.

In other embodiments, the diseases or conditions that may be mediated by modulation of Nav1.7 and/or other voltage-gated sodium channels are substance-related disorders.

In yet other embodiments, the diseases or conditions that may be mediated by modulation of Nav1.7 and/or other voltage-gated sodium channels are Bipolar Disorders (including Bipolar I Disorder, Bipolar II Disorder (i.e., Recurrent Major Depressive Episodes with Hypomanic Episodes) (296.89), Cyclothymic Disorder (301.13) or Bipolar Disorder Not Otherwise Specified (296.80)).

In still other embodiments, the diseases or conditions that may be mediated by modulation of Nav1.7 and other voltage-gated sodium channels are Nicotine-Related Disorders such as Nicotine Dependence (305.1), Nicotine Withdrawal (292.0) or Nicotine-Related Disorder Not Otherwise Specified (292.9).

In some embodiments, the disease or condition is epilepsy, e.g., post-traumatic epilepsy, obsessive compulsive disorders (OCD), sleep disorders (including circadian rhythm disorders, insomnia & narcolepsy), tics (e.g., Giles de la Tourette's syndrome), ataxias, muscular rigidity (spasticity), and temporomandibular joint dysfunction. In other embodiments, the disease or condition is bladder hyperrelexia following bladder inflammation.

In yet other embodiments, the disease or condition is selected from neurodegenerative diseases and neurodegeneration, such as dementia, particularly degenerative dementia (including senile dementia, Alzheimer's disease, Pick's disease, Huntington's chorea, Parkinson's disease and Creutzfeldt-Jakob disease, motor neuron disease). The (5R)-5-(4-{[(2-fluorophenyl)methyl]oxy}phenyl)-L-prolinamide, or a pharmaceutically acceptable salt thereof, may also be useful for the treatment of amyotrophic lateral sclerosis (ALS) or neuroinflamation.

In still other embodiments, the disease or condition is neuroprotection, such as for the inhibition and/or treatment of neurodegeneration following stroke, cardiac arrest, pulmonary bypass, traumatic brain injury, spinal cord injury or the like.

In some embodiments, the disease or condition is tinnitus.

In some embodiments, (5R)-5-(4-{[(2-fluorophenyl)methyl]oxy}phenyl)-L-prolinamide, or a pharmaceutically acceptable salt thereof, is administered in combination with one or more therapeutically effective medicaments. In some such embodiments, the one or more therapeutically effective medicaments comprise an analgesic. For example, such analgesics include for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO 99/012930); 5-lipoxygenase inhibitors; NSAIDs (non-steroidal anti-inflammatory drugs) such as diclofenac, indomethacin, nabumetone or ibuprofen; bisphosphonates; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists or memantine; ligands for the α2δ-subunit of voltage-gated calcium channels, such as gabapentin, pregabalin and solzira; tricyclic antidepressants such as amitriptyline; antiepileptic drugs; cholinesterase inhibitors such as galantamine; mono-aminergic uptake inhibitors such as venlafaxine; opioid analgesics; local anaesthetics; 5HT1 agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP4 receptor ligands; EP2 receptor ligands; EP3 receptor ligands; EP4 agonists and EP2 agonists; EP4 antagonists; EP2 antagonists and EP3 antagonists; cannabinoid receptor ligands; bradykinin receptor ligands; vanilloid receptor or Transient Receptor Potential (TRP) ligands; and purinergic receptor ligands, including antagonists at P2X3, P2X2/3, P2X4, P2X7 or P2X4/7; KCNQ/Kv7 channel openers, such as retigabine; additional COX-2 inhibitors are disclosed in U.S. Pat. Nos. 5,474,995, 5,633,272, 5,466,823, 6,310,099 and 6,291,523; and in WO 96/25405, WO 97/38986, WO 98/03484, WO 97/14691, WO 99/12930, WO 00/26216, WO 00/52008, WO 00/38311, WO 01/58881 and WO 02/18374.

In some embodiments, the methods disclosed herein comprise conjoint administration of (5R)-5-(4-{[(2-fluorophenyl)methyl]oxy}phenyl)-L-prolinamide, or a pharmaceutically acceptable salt thereof, and one or more analgesics (e.g., tramadol or amitriptyline), anticonvulsant drugs (e.g., gabapentin, neurontin or pregabalin (i.e., Lyrica)) or antidepressant drugs (e.g., duloxetine (i.e., Cymbalta) or venlafaxine). The therapeutically effective amount of co-therapy comprising administration of (5R)-5-(4-{[(2-fluorophenyl)methyl]oxy}phenyl)-L-prolinamide, or a pharmaceutically acceptable salt thereof, and at least one suitable analgesic, anticonvulsant or antidepressant drug would be the amount of (5R)-5-(4-{[(2-fluorophenyl)methyl]oxy}phenyl)-L-prolinamide, or a pharmaceutically acceptable salt thereof, and the amount of the suitable analgesic, anticonvulsant or antidepressant drugs that when taken together or sequentially have a combined effect that is therapeutically effective. Further, it will be recognized by one skilled in the art that in the case of conjoint therapy, the amount of (5R)-5-(4-{[(2-fluorophenyl)methyl]oxy}phenyl)-L-prolinamide, or a pharmaceutically acceptable salt thereof, and/or the amount of the suitable analgesic, anticonvulsant or antidepressant drug may or may not be therapeutically effective if administered separately in that amount.

“Administering” or “administration of” a substance, a compound or an agent to a subject can be carried out using one of a variety of methods known to those skilled in the art. For example, a compound or an agent can be administered intravenously, arterially, intradermally, intramuscularly, intraperitoneally, subcutaneously, jocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, a subject who receives such treatment can benefit from a combined effect of different therapeutic agents.

Where (5R)-5-(4-{[(2-fluorophenyl)methyl]oxy}phenyl)-L-prolinamide, or a pharmaceutically acceptable salt thereof, and the analgesic, anticonvulsant or antidepressant agent(s) are administered in separate dosage forms, the number of dosages administered per day for each compound may be the same or different. (5R)-5-(4-{[(2-fluorophenyl)methyl]oxy}phenyl)-L-prolinamide, or a pharmaceutically acceptable salt thereof, and the analgesic, anticonvulsant or antidepressant agent(s) may be administered via the same or different routes of administration. Examples of suitable methods of administration include, but are not limited to, oral, intravenous (iv), intramuscular (im), subcutaneous (sc), intranasal, transdermal, and rectal. (5R)-5-(4-{[(2-fluorophenyl)methyl]oxy}phenyl)-L-prolinamide, or a pharmaceutically acceptable salt thereof, may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. (5R)-5-(4-{[(2-fluorophenyl)methyl]oxy}phenyl)-L-prolinamide, or a pharmaceutically acceptable salt thereof, and the analgesic, anticonvulsant or antidepressant agent(s) may be administered according to simultaneous or alternating regimens, at the same or different times during the course of the therapy, concurrently in divided or single forms.

In one embodiment, (5R)-5-(4-{[(2-fluorophenyl)methyl]oxy}phenyl)-L-prolinamide, or a pharmaceutically acceptable salt thereof, is administered orally.

Also provided herein is (5R)-5-(4-{[(2-fluorophenyl)methyl]oxy}phenyl)-L-prolinamide, or a pharmaceutically acceptable salt thereof, for treating a disease or condition mediated by modulation of Nav1.7, wherein the medicament is for administration to a subject not receiving treatment with a UGT inhibitor.

Also provided herein is use of (5R)-5-(4-{[(2-fluorophenyl)methyl]oxy}phenyl)-L-prolinamide, or a pharmaceutically acceptable salt thereof, for the manufacture of a medicament for treating a disease or condition mediated by modulation of Nav1.7, wherein the medicament is for administration according to a regimen that excludes conjoint treatment with a UGT inhibitor.

Also provided herein is a composition comprising (5R)-5-(4-{[(2-fluorophenyl)methyl]oxy}phenyl)-L-prolinamide, or a pharmaceutically acceptable salt thereof, and a UGT inhibitor, for the manufacture of a medicament for treating a disease or condition mediated by modulation of Nav1.7.

Also provided herein is use of (5R)-5-(4-{[(2-fluorophenyl)methyl]oxy}phenyl)-L-prolinamide, or a pharmaceutically acceptable salt thereof, for treating a disease or condition mediated by modulation of Nav1.7 conjointly with a UGT inhibitor.

The term “subject” as used herein, refer to either a human or a non-human animal. The term “subject” thus includes mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain) is well understood in the art, and includes administration of a composition which reduces the frequency of, or delays the onset of, symptoms of a medical condition in a subject relative to a subject that does not receive the composition. Thus, prevention of a disease or condition mediated by modulation of Nav1.7 includes, for example, reducing the amount of pain experienced by subjects receiving a prophylactic treatment relative to an untreated control population, and/or delaying the pain experienced by subjects in a treated population versus an untreated control population, e.g., by a statistically and/or clinically significant amount.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as pain, e.g., neuropathic pain. The skilled worker can readily determine the effective amount for a given situation by routine experimentation.

(5R)-5-(4-{[(2-fluorophenyl)methyl]oxy}phenyl)-L-prolinamide, or a pharmaceutically acceptable salt thereof, may be administered as the raw chemical but the active ingredient is preferably formulated in a pharmaceutical composition. Thus, in some embodiments, (5R)-5-(4-{[(2-fluorophenyl)methyl]oxy}phenyl)-L-prolinamide, or a pharmaceutically acceptable salt thereof, is administered as a pharmaceutical composition comprising one or more pharmaceutically acceptable carrier(s), diluents(s) and/or excipient(s).

(5R)-5-(4-{[(2-fluorophenyl)methyl]oxy}phenyl)-L-prolinamide may be administered in the form of a pharmaceutically acceptable salt. The pharmaceutically acceptable salt of the compound of formula (I) may be, for example, a non-toxic acid addition salt formed with inorganic acids such as hydrochloric; hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al (1977) J. Pharm Sci. 66, 1-19; P L Gould (1986) International Journal of Pharmaceutics, 33, 201-217; and Bighley et al, Encyclopedia of Pharmaceutical Technology, Marcel Dekker Inc, New York 1996, Volume 13, page 453-497,

In certain embodiments, (5R)-5-(4-{[(2-fluorophenyl)methyl]oxy}phenyl)-L-prolinamide is provided as a hydrochloride salt.

The carrier, diluent, and/or excipient must be “acceptable” in the sense of being compatible with the other ingredients of the composition and not deleterious to the recipient thereof.

As used herein, the term “composition” is intended to encompass a product comprising the specified ingredients in the specified amounts, as well as any product which results, directly or indirectly, from combinations of the specified ingredients in the specified amounts.

Since the (5R)-5-(4-{[(2-fluorophenyl)methyl]oxy}phenyl)-L-prolinamide, or a pharmaceutically acceptable salt thereof, described herein is intended for use in pharmaceutical compositions, it will readily be understood that it is preferably provided in substantially pure form, for example at least 60% pure, more suitably at least 75% pure and preferably at least 85%, especially at least 98% pure (% are given on a weight for weight basis). Impure preparations of (5R)-5-(4-{[(2-fluorophenyl)methyl]oxy}phenyl)-L-prolinamide may be used for preparing the more pure forms used in the pharmaceutical compositions.

Pharmaceutical compositions containing (5R)-5-(4-{[(2-fluorophenyl)methyl]oxy}phenyl)-L-prolinamide, or a pharmaceutically acceptable salt thereof, as the active ingredient can be prepared by intimately mixing (5R)-5-(4-{[(2-fluorophenyl)methyl]oxy}phenyl)-L-prolinamide, or a pharmaceutically acceptable salt thereof, with a pharmaceutical carrier, e.g., according to conventional pharmaceutical compounding techniques. These procedures may involve mixing, granulating and compressing or dissolving the ingredients as appropriate to the desired preparation.

The (5R)-5-(4{[(2-fluorophenyl)methyl]oxy}phenyl)-L-prolinamide, or a pharmaceutically acceptable salt thereof, may be administered in conventional dosage forms prepared by combining (5R)-5-(4{[(2-fluorophenyl)methyl]oxy}phenyl)-L-prolinamide, or a pharmaceutically acceptable salt thereof, with standard pharmaceutical carriers or diluents according to conventional procedures well known in the art. These procedures may involve mixing, granulating and compressing or dissolving the ingredients as appropriate to the desired preparation.

(5R)-5-(4{[(2-fluorophenyl)methyl]oxy}phenyl)-L-prolinamide, or a pharmaceutically acceptable salt thereof, may be administered by any suitable method, e.g., by oral, parenteral, buccal, sublingual, nasal, rectal or transdermal administration, and the pharmaceutical compositions adapted accordingly, for administration to mammals including humans. In some embodiments, the (5R)-5-(4-{[(2-fluorophenyl)methyl]oxy}phenyl)-L-prolinamide or salt thereof is administered orally.

(5R)-5-(4{[(2-fluorophenyl)methyl]oxy}phenyl)-L-prolinamide, or a pharmaceutically acceptable salt thereof can be formulated as liquids or solids, e.g., as syrups, suspensions, emulsions, tablets, capsules or lozenges.

The topical formulations of the present invention may be presented as, for instance, ointments, creams or lotions, eye ointments, and eye or ear drops, impregnated dressings and aerosols, and may contain appropriate conventional additives such as preservatives, solvents to assist drug penetration, and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

A liquid formulation will generally consist of a suspension or solution of the active ingredient in a suitable liquid carrier(s), e.g., an aqueous solvent, such as water, ethanol or glycerine, or a non-aqueous solvent, such as polyethylene glycol or an oil. The formulation may also contain a suspending agent, preservative, flavouring and/or colouring agent.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non aqueous vehicles (which may include edible oils), for example almond oil, oily esters such as glycerine, propylene glycol, or ethyl alcohol; preservatives, for example methyl or propyl p hydroxybenzoate or sorbic acid, and, if desired, conventional flavouring or colouring agents.

Typical parenteral compositions consist of a solution or suspension of the active ingredient in a sterile vehicle, water being preferred, or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration. (5R)-5-(4-{[(2-fluorophenyl)methyl]oxy}phenyl)-L-prolinamide, or a pharmaceutically acceptable salt thereof, depending on the vehicle and concentration used, can be either suspended or dissolved in the vehicle. In preparing solutions (5R)-5-(4-{[(2-fluorophenyl)methyl]oxy}phenyl)-L-prolinamide, or a pharmaceutically acceptable salt thereof, can be dissolved in water for injection and filter-sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that (5R)-5-(4-{[(2-fluorophenyl)methyl]oxy}phenyl)-L-prolinamide, or a pharmaceutically acceptable salt thereof, is suspended in the vehicle instead of being dissolved and sterilisation cannot be accomplished by filtration. (5R)-5-(4-{[(2-fluorophenyl)methyl]oxy}phenyl)-L-prolinamide, or a pharmaceutically acceptable salt thereof, can be sterilised by exposure to ethylene oxide before suspending in the sterile vehicle. Advantageously, a surfactant or wetting agent is included in the composition to facilitate uniform distribution of (5R)-5-(4-{[(2-fluorophenyl)methyl]oxy}phenyl)-L-prolinamide, or a pharmaceutically acceptable salt thereof.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant that can be a compressed gas, e.g., air, or an organic propellant such as a fluoro-chloro-hydro-carbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin. Compositions for rectal administration are conveniently in the form of suppositories containing a conventional suppository base such as cocoa butter. Compositions suitable for transdermal administration include ointments, gels and patches.

In some embodiments, the composition is in unit dose form such as a tablet, capsule or ampoule.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Materials and Methods

### Study Design

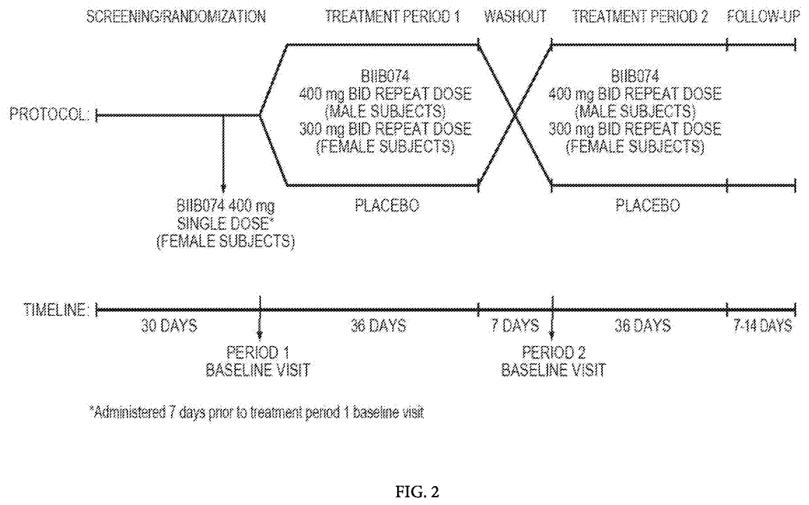

This was a Phase 1, randomized, double-blind, placebo-controlled, repeat dose, 2-period cross-over study to investigate the effect of BIIB074 300-400 mg bid on ambulatory blood pressure (ABP) in healthy participants (FIG. 2). The study comprised: screening (to occur a maximum of 30 days before the first baseline assessment); two 36-day treatment periods, each preceded by a baseline visit and separated by a 7-day washout (to minimize possible carry over effects); and a follow-up period of 7-14 days after last dose. Prior to this study, no females had received BIIB074; for this reason, a single dose BIIB074 session at the dose level of 400 mg was also conducted in female participants one week prior to the period 1 baseline visit. Following this session, some participants were predicted to exceed the predefined PK limit (area under the plasma concentration-time curve [AUC] 97 μg·h/mL) when receiving 400 mg bid at steady state. Therefore, in the subsequent phases of the study, all female participants received a lower dose of 300 mg bid (males received 400 mg bid).

The study was conducted at one clinical site (Buffalo Clinical Research Center) in the United States. All participants provided written informed consent. The study protocol, participant information and informed consent forms were reviewed and approved by relevant independent ethics committees or institutional review boards, and the study was conducted in accordance with the International Conference on Harmonization principles of Good Clinical Practice and principles of the Declaration of Helsinki.

### Study Population

Eligible participants were healthy males or females between the ages of 18-65 years. The following additional criteria applied for eligibility: body weight>50 kg; body mass index (BMI) within the range 19-40.0 kg/m²; no significant abnormalities on clinical examination, clinical chemistry, or hematology parameters; non-child bearing potential or willing to use agreed methods of contraception.

Volunteers had to abstain from taking prescription or non-prescription drugs within 7 days (or 14 days if the drug was a potential enzyme inducer) or 5 half-lives, whichever was longer, prior to the first dose of study medication until completion of the follow-up visit, unless in the opinion of the investigator and sponsor the medication would not interfere with the study.

### Randomization and Masking

Participants were assigned to treatment sequences in accordance with a randomization schedule generated by Discovery Biometrics, prior to the start of the study, using validated software. Study treatment was BIIB074 400 mg bid for males/300 mg bid for females, or placebo, for 36 days. Prior to dosing, volunteers were randomized into one of the following treatment sequences, BIIB074 (period 1):placebo (period 2) or placebo (period 1):BIIB074 (period 2), and more specifically AB and BA if male and CAB and CBA if female, where A=placebo, B=BIIB074 400 mg bid in males and 300 mg bid in females, C=BIIB074 400 mg single dose in females. Randomization numbers were assigned by the site ensuring there was a balance of sequences (AB/BA and CAB/CBA) in each group. Periods 1 and 2 were double-masked to patients and study personnel.

### Study Medication

BIIB074 was supplied as film-coated, brownish yellow, oblong, biconvex tablets in two strengths: 150 mg and 200 mg. Placebo tablets visually matched the active tablets. All tablets were taken orally with 240 mL of water.

### Outcomes

The primary endpoint was change in 24 h average SBP and DBP from baseline to day 36 as determined by ABPM. Secondary outcome measures included: change in 24 h average SBP and DBP from baseline to days 4 and 15; change in average SBP and DBP within a 12 h dosing interval from baseline to days 14 and 35 (inpatient); change in 24 h average ambulatory heart rate from baseline to days 4, 15 and 36; proportion of participants whose 24 h SBP and DBP increased by <5, 5-9, 10-14, 15-19, and >20 mm Hg compared with baseline; PK parameters of BIIB074 following a single oral dose in healthy female participants, and following repeated oral doses given twice daily to healthy male and female participants; PK/pharmacodynamic (PD) analyses to examine the correlation between ABP and plasma levels and/or metrics of the systemic exposure of BIIB074.

ABP was collected over 24 hours on an outpatient basis at baseline and at days 4, 15 and 36, and over 12 hours on an inpatient basis at baseline, and at days 14 and 35. The ABPM device was placed on the non-dominant arm (except in clinical situations that prohibited measuring BP in the non-dominant arm). BP and heart rate were measured every 15 minutes.

Safety was assessed through monitoring of adverse events (AEs), vital signs, electrocardiogram (ECG), and laboratory safety tests (including clinical chemistry).

### Statistical Analysis

Non-inferiority was based on the one-sided 95% confidence interval (CI) for BIIB074-placebo excluding an effect of >5 mmHg in SBP or DBP. It was planned to recruit approximately 60 participants in order to obtain a minimum of 48 evaluable for ABPM during the repeat dose phase, for at least 90% power, assuming a within-subject standard deviation (SDw) of 8.21 mmHg.

ABPM data were analyzed using a repeated measures mixed effects model, whereby fixed effects were treatment, day, treatment*day, period, average baseline*day, period adjusted baseline*day, sex and treatment*sex; random effect was subject; and repeated effect was day. All summary statistics were carried out using SAS 8.02 for UNIX running under the Harmonisation of Analysis and Reporting Program (HARP) environment. PK parameters were calculated by standard non-compartmental analysis according to working practices and using Win Nonlin Pro v. 5.2.

The safety population was the primary analysis population for this study and included all participants who received one or more doses of BIIB074. The PK population was defined as participants in the safety population for whom a PK sample was obtained and analyzed.

### Example 1: (5R)-5-(4-{[(2-Fluorophenyl)methyl]oxy}phenyl)-L-prolinamide hydrochloride (E1; Also Referred to Herein and/or Known as Vixotrigine, Raxatrigine, BIIB074, GSK1014802 and CNV1014802)

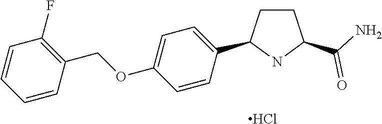 [SMILES: NC(=O)[C@@H]1CC[C@H](c2ccc(OCc3ccccc3F)cc2)N1 ; Cl]

(5R)-5-(4-{[(2-fluorophenyl)methyl]oxy}phenyl)-L-prolinamide of Example 1 may be prepared as described in Example 2, Procedures 1 to 5 of U.S. Pat. No. 7,655,693.

### Example 2: Dose Selection Methodology

Selection of the 150 and 250 mg TID dose of the present invention was based on three different criteria: efficacy in preclinical models of pain, comparison with the 350 mg BID dose which demonstrated clinical benefit in a painful lumbosacral radiculopathy Phase 2 study, and comparison with efficacious doses of marketed drugs in trigeminal neuralgia, using an in vitro assay to quantify activity at the primary target hNav1.7.

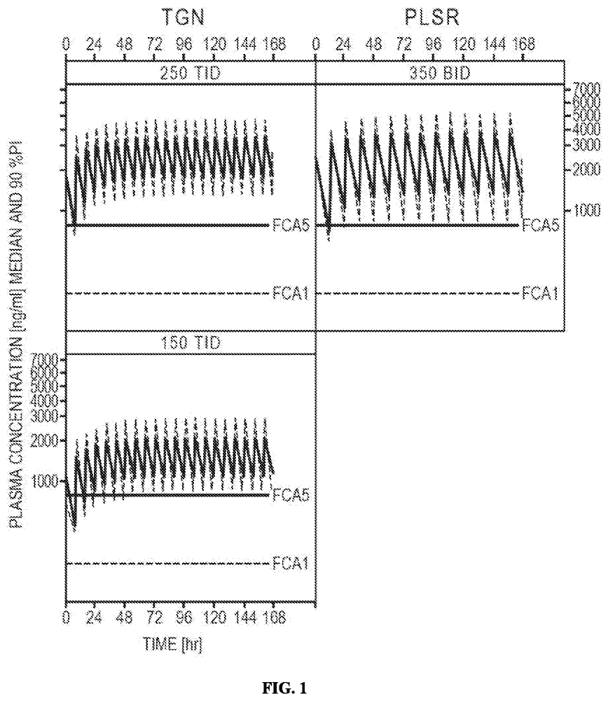

At steady stated, the C_{Trough} exposure of Example 1 at the low dose of 150 mg TID and the high dose of 250 mg TID (1099 ng/ml and 1750 ng/ml, respectively) is higher than the human scaled equivalent total plasma exposure of 786 ng/ml where a robust efficacy was observed in a rat model of inflammation (see FIG. 1). In this model, inflammation was induced by intraplantar injection of Freud Complete Adjuvant.

Mechanical hypersensitivity was then assessed using weight bearing. The oral dose of 1 mg/kg was identified as the minimal effective dose and 5 mg/kg fully reversed the mechanical hypersensitivity.

From the PK modelling plots, the C_{Max} for 250 mg TID was equivalent to that of another dose, 350 mg BID (Table 1), which has demonstrated clinical benefit in a Phase 2 study in patients with lumbosacral radiculopathy (A novel proof of concept, randomized, double blind, cross-over study, demonstrating the safety and efficacy of CNV1014802 in subjects with neuropathic pain from lumbosacral radiculopathy, American Pain Society meeting, Palm Springs, 2015).

TABLE 1
Comparison of activity of clinical anticonvulsants and Example 1 at several doses:
The levels of inhibition (% inhibition) are extracted form the Example 1 dose-response plots
at mid point inactivation for each Nav subtype. The exposures for Example 1 are extracted
from dose modelling plots and the exposures/doses for marketed anticonvulsants have been
found in various sources of literature below.

| | | Free plasma | Human Naᵥ1.7 |
|---|---|---|---|
| Cₘₐₓ | Free fraction | (μM) | % Inhibition |
| ^{1,2}Carbamazepine (TGN) 200 mg | 24% | 4-12 | 11-38 |
| qid | | | |
| (4-12 μg/ml total plasma) | | | |
| EFFICACY OBSERVED | | | |
| Example 1 350 mg bid | 3.2% | 0.37 | 38 |
| (3.74 μg/ml total plasma) | | | |
| EFFICACY OBSERVED in | | | |
| PLSR | | | |
| Example 1 200 mg bid | 3.6% | 0.29 | 40 |
| (2.8 μg/ml total plasma) | | | |
| EFFICACY EXPECTED in | | | |
| PLSR | | | |
| Example 1 250 mg tid | 3.2% | 0.36 | 38 |
| (3.43 μg/ml total plasma) | | | |
| EFFICACY EXPECTED in TN | | | |
| Example 1 150 mg tid | 3.2% | 0.21 | 31 |
| (2.06 μg/ml total plasma) | | | |
| EFFICACY OBSERVED in TN | | | |
| ^{3, 4}Lamotrigine (TN) 200 mg bid | 44% | 5.8 | 6 |
| (3.4 μg/ml total plasma) | | | |
| NO EFFICACY OBSERVED | | | |

¹Wiffen et al (2014) Carbamazepine for chronic neuropathic pain and fibromyalgia in adults. Cochrane Database of Systematic Reviews, Issue 4.
²Prescribing information Carbamazepine, https://www.pharma.us.novartis.com/product/pi/pdf/tegretol.pdf, Sept 2015
³Wiffen et al (2013) Lamotrigine for chronic neuropathic pain and fibromyalgia in adults. Cochrane Database of Systematic Reviews, Issue 12.
⁴Rambeck B and Wolf P. (1993) Lamotrigine clinical pharmacokinetics. Clinical Pharmacokinetics, 25(6):433-43.

The C_{Trough} for 250 mg TID is higher than that of the 350 mg BID which is another reason for selection of this dose.

In Table 1, free plasma C_{Max} exposures of Example 1 obtained from modelling different dosing regimens were used to quantify the resulting amount of block at the primary target hNaV1.7. In the assay chosen for comparison purposes, at the doses of 250 mg TID, 350 mg TID and 150 mg TID, the inhibitions at NaV1.7 are 38, 38% and 31%, respectively. Doses of marketed drugs used in trigeminal neuralgia were compared using the same paradigm. The amount of inhibition of hNaV1.7 obtained with Example 1 is in the range of activity obtained with the best exposures of carbamazepine used at 200 mg QID (11 to 38% inhibition), and much higher than exposures obtained with lamotrigine used at 200 mg bid (6% inhibition), which shows little or no efficacy in trigeminal, providing confidence on favourable outcome on efficacy.

The convergence of preclinical and clinical evidence on Example 1 provided the rationale to select the new dose of 250 mg TID for trigeminal neuralgia.

### Example 3: 150 mg TID Dosage Study

A clinical trial was conducted to evaluate certain pharmacokinetic parameters of the compound of Example 1 when dosed at 150 mg TID for seven days. 15 young males and females aged 18 to 45 were scheduled to received ether the compound of Example 1 at 150 mg TID during a first period of 8 days followed by placebo during a second period of 8 days; or placebo during the first period and the compound of Example 1 during the second period.

The subjects were exhibited the following pharmacokinetic parameters on day 8 of the period during which they received the compound of Example 1: AUC₀₋₈ (h·ng/mL)=15319 (20.6); Cₘₐₓ (ng/mL)=2711 (21.0); Cₘᵢₙ=1313 (25.7).

### Example 4: 300/400 mg BID Dosage Study

This study reports the results of a Phase 1, randomized cross-over trial designed to assess inpatient and outpatient ambulatory blood pressure monitoring (ABPM) in healthy volunteers treated with the compound of Example 1 (BIIB074) for 36 days.

### Results

The first participant was enrolled in the study on Jul. 13, 2009 and the last participant completed on Dec. 21, 2009. Overall, 60 participants were enrolled, of whom 10 withdrew prematurely (7 due to an AE, 2 at the investigator's discretion, and 1 withdrew consent).

The mean age of the overall population (n=60) was 34.3 years and 40% were female. Participants' baseline demographics are summarized in Table 2. Mean duration of treatment with BIIB074 (300-400 mg bid repeat dosing) was 35.4 days, and mean dose of BIIB074 was 361.1 mg. Mean duration of treatment with placebo was 34.4 days.

TABLE 2
Baseline demographics

| | All participants | | |
|---|---|---|---|
| Demographics | N = 60 | | |
| Age in years, mean (SD) | 34.3 (11.63) | | |
| Sex, n (%) | | | |
| Female | 24 (40) | | |
| Male | 36 (60) | | |
| BMI in kg/m², mean (SD) | 27.07 (4.009) | | |
| Height in cm, mean (SD) | 169.9 (9.23) | | |
| Weight in kg, mean (SD) | 78.35 (13.679) | | |
| | | BIIB074 | BIIB074 |
| | | 300-400 mg | 400 mg |
| Baseline vital signs | Placebo | repeat dose | single dose |
| at Day 1, predose* | N = 54 | N = 54 | N = 22 |
| SBP in mmHg, mean (SD) | 116.9 (11.78) | 117.1 (11.03) | 114.8 (10.66) |
| DBP in mmHg, mean (SD) | 78.6 (7.77) | 77.5 (7.85) | 78.5 (4.56) |
| Heart rate in beats/min, | 79.5 (13.00) | 78.3 (12.20) | 81.9 (12.50) |
| mean (SD) | | | |

N, number of participants; BMI, body mass index; SD, standard deviation; SBP, systolic blood pressure; DBP, diastolic blood pressure.
*Vital signs were recorded on Day 1 of each treatment period, predose time and standing position. Data should be interpreted with caution given the cross-over design and potential for carryover between treatment periods despite washouts.

### Ambulatory Blood Pressure Monitoring

No participant had changes of BP or heart rate meeting the flagging criteria defined in the protocol or that were considered clinically significant by the investigator.

### Outpatient ABPM

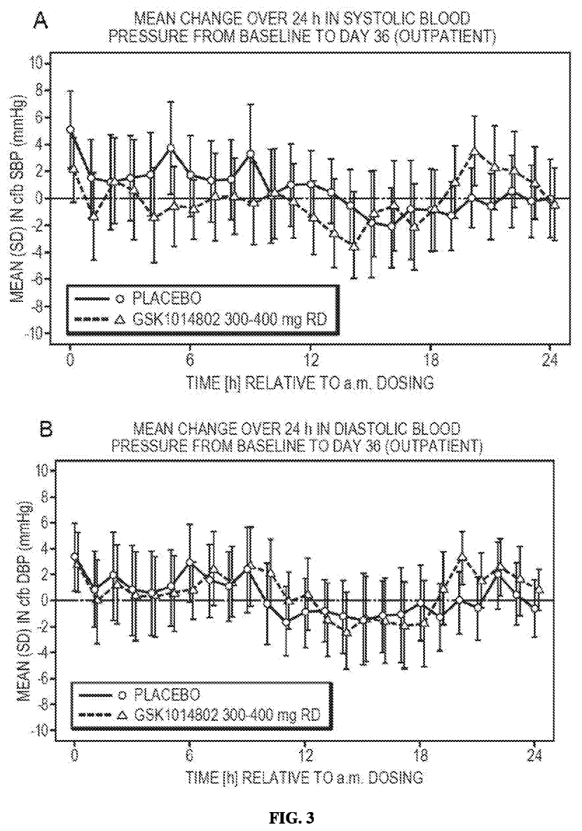

Changes from baseline in hourly BPs over 24 hours at the end of the 36 day period are shown in FIG. 3. These data demonstrate that BIIB074 had similar effects on 24 h BP to placebo.

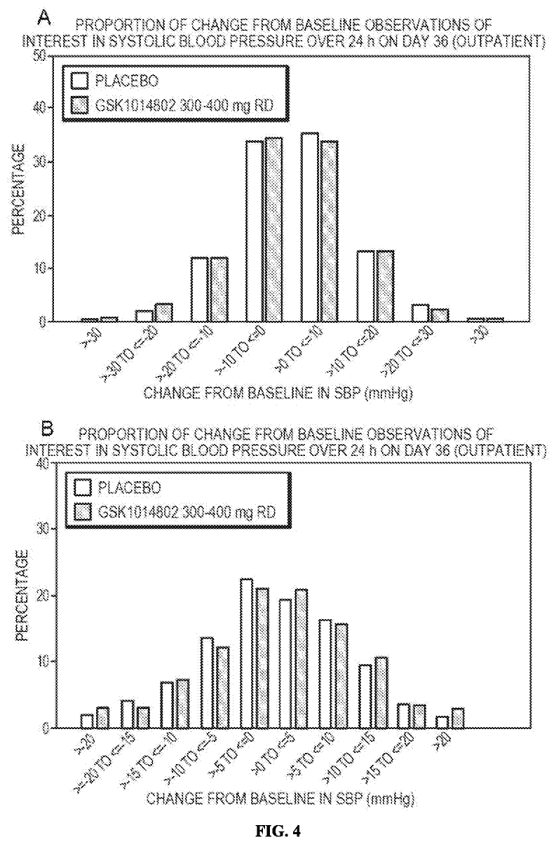

In addition to assessment of the mean changes from baseline in 24 h BP, it is relevant to study the range of individual changes on drug versus placebo to determine if there may be a small percentage of substantial outliers. Examination of change from baseline in outpatient 24 h SBP and DBP revealed a normal distribution (FIG. 4), with the majority of SBP and DBP measurements at day 36 within 0-10 mmHg of their associated time-matched baseline for both treatments. There was no evidence to suggest a significant increase in SBP or DBP for BIIB074.

Additionally, a clinically relevant effect was considered to be >20% of participants on BIIB074 having an average 24 h increase from baseline in SBP>30 mmHg or DBP>20 mmHg versus placebo. 4/1249 observations (0%) fell in the category SBP>30 mmHg at day 36 for BIIB074 (versus 4/1072 observations [0%] for placebo) (FIG. 4). Also, 35/1249 observations (3%) fell in the category DBP>20 mmHg at day 36 for BIIB074 (versus 19/1072 observations [2%] for placebo) (FIG. 4).

Summaries and mixed model repeated measures outputs of the analyses of outpatient 24 h SBP and DBP on days 4, 15 and 36, over 24 h are provided in Table 3. Mean change in average SBP from baseline to day 36 was −0.327. Non-inferiority of BIIB074 compared to placebo was demonstrated for outpatient 24 h SBP and DBP since the one-sided 95% CI for BIIB074-placebo excluded an effect>5 mmHg. In fact, due to very low within-subject variability observed in these normal healthy participants (SDw=3.8 mmHg for SBP and SDw=2.9 mmHg for DBP), the power of the study was larger than planned and a smaller effect size than 5 mmHg could be ruled out. The upper bound of the one-sided 95% CI also was <2 mmHg for the majority of SBP and DBP comparisons on days 4, 15, and 36 with the exception of SBP on day 4 (˜2.2 mmHg).

TABLE 3
Summary of the Analysis of Change in 24 h Average SBP and DBP from Baseline
to Days 4, 15 and 36 (Outpatient)

| | | LS Mean | | | |
|---|---|---|---|---|---|
| | Visit | BIIB074 | LS Mean | BIIB074- | 90% CI for |
| Parameter | (Day) | 300-400 mg | Placebo | Placebo | Difference* |
| SBP | 4 | 2.372 | 1.366 | 1.006 | (−0.226, 2.238) |
| (mmHg) | 15 | 0.915 | 0.621 | 0.294 | (−0.929, 1.516) |
| | 36 | −0.327 | 0.180 | −0.507 | (−1.755, 0.741) |
| DBP | 4 | 1.856 | 1.066 | 0.789 | (−0.258, 1.837) |
| (mmHg) | 15 | 0.907 | 0.262 | 0.645 | (−0.395, 1.685) |
| | 36 | 0.201 | −0.086 | 0.287 | (−0.776, 1.350) |

LS, least square; SBP, systolic blood pressure; DBP, diastolic blood pressure; CI, confidence interval.
*Two-sided 90% CI equates to a one-sided 95% CI.

To further explore the potential occurrence of clinically relevant changes in BP, the proportions of participants whose BP increased by more than 10 mmHg from baseline and who had a resultant absolute value>130 mmHg for SBP or >80 mmHg for DBP were calculated. On day 36, 6.0% of BP values on placebo and 5.0% of observations on BIIB074 fell into this category for SBP, while 6.3% of observations on placebo and 6.9% of observations on BIIB074 fell into this category for DBP (Table 4).

TABLE 4
Proportion of Observations with Changes in SBP >10 mmHg that Caused
Shift into Hypertensive Range (SBP >130 mmHg and DBP >80 mmHg) over 24 h for
Days 4, 15 and 36 (Outpatient)

| | | | BIIB074 |
|---|---|---|---|
| Visit | | Placebo | 300-400 mg repeat |
| (Day) | | (N = 54) | dose (N = 54) |
| 4 | n | 1197 | 1294 |
| | SBP: Change >10 and absolute value >130 mmHg | 92 (7.7%) | 116 (9.0%) |
| | DBP: Change >10 and absolute value >80 mmHg | 103 (8.6%) | 137 (10.6%) |
| 15 | n | 1199 | 1322 |
| | SBP: Change >10 and absolute value >130 mmHg | 72 (6.0%) | 95 (7.2%) |
| | DBP: Change >10 and absolute value >80 mmHg | 77 (6.4%) | 109 (8.3%) |
| 36 | n | 1072 | 1249 |
| | SBP: Change >10 and absolute value >130 mmHg | 64 (6.0%) | 63 (5.0%) |
| | DBP: Change >10 and absolute value >80 mmHg | 67 (6.3%) | 86 (6.9%) |

N, number of participants; n, number of observations; SBP, systolic blood pressure; DBP, diastolic blood pressure.

### Inpatient ABPM

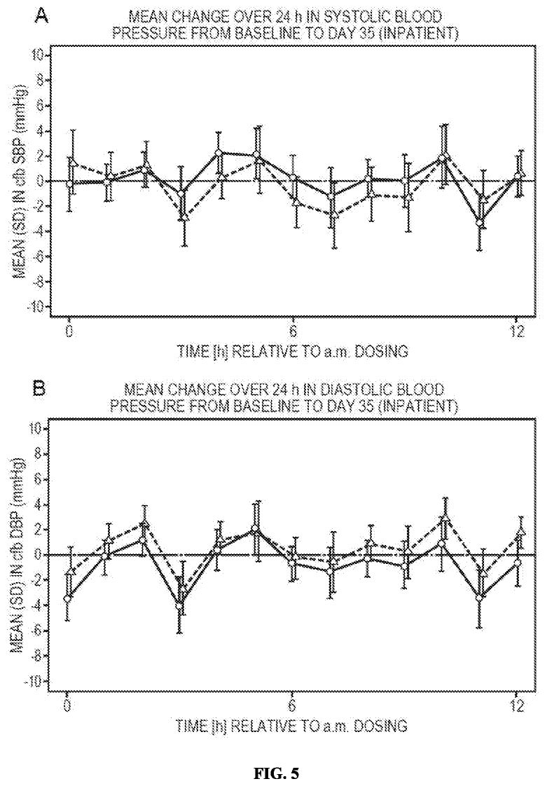

Analysis of the inpatient 12 h ABPM showed very similar findings to the ABPM data captured over 24 hours as an outpatient (FIG. 5). There were no significant differences between BIIB074 and placebo after 36 days of therapy. Similar to the outpatient ABPM results, the majority of inpatient 12 h SBP and DBP measurements at day 35 were within 0-10 mmHg of their associated time-matched baseline for both treatments. No observations showed an increase of ≥30 mmHg for SBP and only a few observations showed an increase of ≥20 mmHg for DBP.

In contrast to the outpatient ABPM readings, the inpatient ABPM measurements demonstrated a slight increase in change from baseline (2.0-2.5 mmHg/bpm) at days 14 and 35 for SBP, DBP, and heart rate; however, this was not considered clinically meaningful and non-inferiority of BIIB074 compared with placebo was demonstrated, since the one-sided 95% CI for the difference BIIB074-placebo excluded an effect≥5 mmHg.

### Safety

The most common AEs during BIIB074 treatment were nervous system disorders such as headache and dizziness, followed by nasopharyngitis, nausea and vomiting. The rate of AEs was generally very similar to placebo, particularly for the most common AE of headache (n=11 [20%] for BIIB074 300-400 mg bid repeat dose versus n=10 [19%] for placebo). The majority of AEs associated with BIIB074 300-400 mg bid repeat dose were mild in nature, apart from 9 AEs of moderate intensity (headache, dizziness, 2× oropharyngeal pain, nasal congestion, ulcer hemorrhage [verbatim: “hemorrhagic ulcerations on lips”], neck pain, eye pain, abnormal liver function test) and 2 AEs of severe intensity (headache, oral disorder [verbatim: “oral lesions”]). All AEs associated with BIIB074 400 mg single dose in females were mild in nature. Table 6 summarizes AEs that occurred in >2 participants in any treatment group.

Out of the ten (17%) participants who were withdrawn from the study, 7 (12%) were due to AEs (2 were on placebo and 5 on BIIB074 at the time of withdrawal). For 1 participant on placebo, the AE started prior to dosing. One of the withdrawals was due to erythema multiforme (with hemorrhagic mouth ulcers) in a participant who had received BIIB074. No serious AEs were reported in this study. There were no clinically significant ECG changes in either treatment group, and the majority of ECGs from day 1-35 were normal. There were no changes in clinical laboratory values that were considered to be of clinical importance.

TABLE 6
Adverse events occurring in ≥2 participants
in any treatment group

| | | BIIB074 300- | |
|---|---|---|---|
| | | 400 mg bid | BIIB074 400 mg |
| | Placebo | repeat dosing | single dose |
| | N = 54 | N = 54 | N = 22 |
| Preferred Term | n (%) | n (%) | n (%) |
| Participants with | 26 (48) | 25 (46) | 12 (55) |
| any AE | | | |
| Headache | 10 (19) | 11 (20) | 6 (27) |
| Dizziness | 3 (6) | 6 (11) | 5 (23) |
| Nausea | 2 (4) | 4 (7) | 3 (14) |
| Vomiting | 2 (4) | 3 (6) | 1 (5) |
| Diarrhoea | 3 (6) | 1 (2) | 0 |
| Nasopharyngitis | 5 (9) | 6 (11) | 0 |
| Oropharyngeal | 1 (2) | 2 (4) | 0 |
| pain | | | |
| Pyrexia | 1 (2) | 2 (4) | 0 |
| Fatigue | 0 | 2 (4) | 0 |
| Pain in extremity | 2 (4) | 0 | 0 |
| Rash | 3 (6) | 1 (2) | 0 |
| Hypersensitivity* | 0 | 2 (4) | 0 |

AE, adverse event; bid, twice daily.
*Verbatim text: allergy symptoms.

### Pharmacokinetics

Following single dose administration to female participants, BIIB074 was characterized by rapid and extensive absorption (plasma concentrations were measurable in all female participants between 0.5 and 24 h). Peak levels were achieved within 1.5 h post-dosing and, afterwards, plasma levels declined with a median terminal half-life (t_{1/2}) of ˜9 h (Table 7). AUC over the 24 h dosing interval [AUC₍₀₋₂₄₎] were characterized by a small between-subject variability (coefficient of variation between subjects [CV %] 20-25%). AUC₍₀₋₂₄₎ in males receiving BIIB074 repeat dose at a dose level of 400 mg bid was on average 10% higher than in females receiving the same compound at a dose level of 300 mg bid, on days 14 and 35. In the same conditions, maximum observed concentration (Cₘₐₓ) in males was on average 11-19% higher than in females. Following repeat dosing (days 14 and 35), dose-normalized AUC and Cₘₐₓ were, on average, 17-18% and 11-17% lower in male than in female participants (Table 7), likely due to a dependency of BIIB074 exposure on body size.

TABLE 7
BIIB074 Pharmacokinetic Parameters

| | Single Dose | Repeat dose (females: 300 mg bid; males: 400 mg bid) | | | | | |
|---|---|---|---|---|---|---|---|
| | (400 mg) | Day 1 | | Day 14 | | Day 35 | |
| | Female | Female | Male | Female | Male | Female | Male |
| PK Parameter* | (N = 22) | (N = 21) | (N = 33) | (N = 21) | (N = 33) | (N = 21) | (N = 33) |
| AUC₍₀₋₁₂₎ | 24200 (20.9) | 16200 (20.4) | 19100 (19.9) | 29200 (24.7) | 32100 (23.5) | 27400 (23.4) | 30100 (21.8) |
| (ng · h/mL)* | | | | | | | |
| AUC₍₀₋₂₄₎ | 48300 (20.9) | 32400 (20.4) | 38300 (19.9) | 58300 (24.7) | 64100 (23.5) | 54800 (23.4) | 60100 (21.8) |
| (ng · h/mL)* | | | | | | | |
| Cₘₐₓ (ng/mL)* | 3780 (20.4) | 2570 (22.2) | 3210 (22.1) | 4030 (21.2) | 4790 (24.1) | 3990 (26.6) | 4410 (21.6) |
| tₘₐₓ (h)^{†} | 1.50 | 1.50 | 1.00 | 1.50 | 1.00 | 1.00 | 1.00 |
| | (0.50, 3.0) | (0.50, 3.0) | (0.50, 3.0) | (1.00, 3.0) | (0.50, 2.5) | (0.50, 2.5) | (0.50, 3.0) |
| C₁₂ₕ (ng/mL)* | ND | 746 (20.8) | 889 (23.6) | 1440 (29.9) | 1590 (28.3) | 1310 (24.0) | 1460 (26.4) |
| AUC₍₀₋ₜ₎ | 32800 (21.9) | ND | ND | ND | ND | ND | ND |
| (ng · h/mL)* | | | | | | | |
| AUC_{(0-∞)} | 38700 (24.1) | ND | ND | ND | ND | ND | ND |
| (ng · h/mL)* | | | | | | | |
| AUCₑₓ (%)^{†} | 15.2 | ND | ND | ND | ND | ND | ND |
| | (8.70, 26.6) | | | | | | |
| t_{1/2} (h) | 8.91 (13.7) | ND | ND | ND | ND | ND | ND |

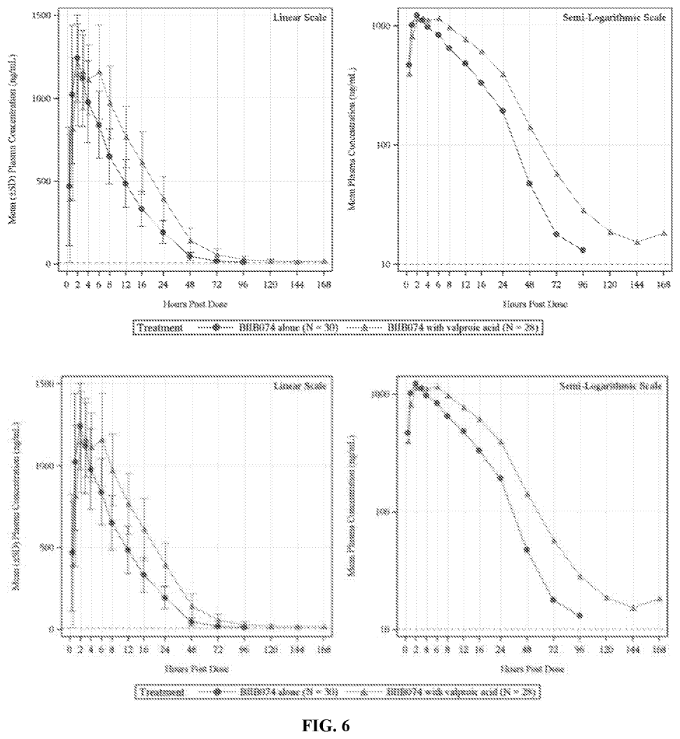

PK/PD analyses of ABPM inpatient data (for which observed plasma concentrations were available) indicated a statistically significant but minimal linear increase of DBP and SBP with increasing BIIB074 observed plasma concentrations (FIG. 6). The slopes of the linear relationships were small (approximately 0.00077±0.00012 and 0.00056±0.00013 mmHg/(ng/mL)), indicating, on average, an increase of DBP and SBP of less than 3 and 2 mmHg, respectively, over the 24 h interval.

### Discussion

Based on the overall results of this study, it was concluded that outpatient and inpatient ABPM were consistent in demonstrating a lack of clinically relevant change in SBP and DBP following repeat doses of BIIB074 for 36 days. Non-inferiority was demonstrated since the 2-sided 90% CI (1-sided 95% CI) for BIIB074-placebo excluded an effect of 5 mmHg for outpatient and inpatient systolic and diastolic BP. PK/PD analyses of ABPM inpatient data indicated a small increase of DBP and SBP with increasing BIIB074 observed plasma concentrations. However, this analysis suggested that the increase was lower than 3 and 2 mmHg for DBP and SBP, respectively, and was not considered clinically relevant.

BIIB074 was well tolerated in this study, with most AEs mild to moderate. The most common AEs during BIIB074 treatment were headache and dizziness, occurring with a rate similar to placebo. AEs were also consistent with earlier Phase 1 studies (single and multiple ascending dose) in healthy male volunteers (Data on File), and Phase 2 studies in TN (Tate et al. (2015) American Pain Society—34th Annual Scientific Meeting. 16(4): S72[386]) and PLSR (Tate et al. (2015) American Pain Society—34th Annual Scientific Meeting. 16(4): S72[387]). One participant reported skin rash of erythema multiforme, which was considered to be related to BIIB074. Because allergic skin reactions have been observed with other sodium channel blockers (e.g., lamotrigine), future studies will continue to closely monitor for occurrence of serious rash.

Ambulatory BP monitoring is a more robust means than clinic measurements to evaluate destabilization of BP values on a non-cardiac drug (White et al. (2002) Hypertension 39(4): 929-934). The use of ABPM in this study has the advantage of providing BP readings when subjects are in their own environment (outpatient), which is regarded in the field as more representative of change as opposed to a clinic setting. Additional benefits of ABPM include: 1) non-invasiveness for the monitored subjects; 2) superior reliability (over 24 h) compared with a one-off measurement; 3) higher value (more accurate) in the overall assessment of cardiovascular risk and severity of hypertension (Mancia and Verdecchia (2015) Circulation Research 116(6): 1034-1045). Hence, it is believed that the results seen in the 54 participants who completed this trial outweigh those from the earlier Phase 1 studies that indicated possible BP effects (data not shown).

The 36 day treatment duration in this study was rationally designed to determine whether tolerance developed for any potential effects of BIIB074 on SBP or DBP, since BP effects resolved by day 28 in the earlier phase 1 trial. The data from this study show a slight trend towards a decrease in the BP difference between BIIB074 and placebo between day 4 and day 35, although differences were minimal at all time points. An additional point of note is that, in preclinical safety/pharmacology studies, there were no effects of BIIB074 on cardiovascular parameters in dogs, and no effects on tyramine-induced hypertension in rats (data not shown). Thus, the body of evidence, encompassing clinical and preclinical studies, supports safety and minimal effects of BIIB074 on BP/cardiovascular parameters.

Limitations of the current study were the relatively small population and the short-term duration of treatment and BP assessment (36 days). It should also be considered that the investigation was carried out among healthy individuals rather than the intended patient population with neuropathic pain and associated comorbidities. The current study population was also younger (mean age 34.3 years) than the intended patient population; for instance, peak onset age of TN is between 50-60 years (Cruccu et al. (2008) Eur J Neurol 15(10): 1013-1028); and for PLSR, individuals are most likely to develop symptoms between 40-60 years of age (Tarulli and Raynor (2007) Neurol Clin 25(2): 387-405). There are additionally some inherent limitations with current ABPM devices, which only record intermittent BP readings (every 15 minutes) of the entire 24-h BP profile, compared to an ideal futuristic beat-to-beat ambulatory BP device (Mancia and Verdecchia (2015) Circulation Research 116(6): 1034-1045).

Overall, despite these limitations, the results from this study confirm that it is unlikely that a clinically important hypertensive signal will be observed for BIIB074 in normotensive subjects, and it is believed that monitoring of the in-clinic BP does not need to be carried out in larger studies.

### Example 5: Phase 1, Open-Label, Fixed-Sequence Study to Evaluate the Effect of UGT Inhibition by Valproic Acid on the Pharmacokinetics of BIIB074 in Healthy Subjects

In human hepatocytes, BIIB074 is mainly metabolized by uridine diphosphate glucuronosyltransferases (UGTs). Based upon the clinical studies conducted to date, metabolism of BIIB074 by UGTs in humans produces 2 glucuronide metabolites: M13 (N carbamoyl glucuronide, CNV3000497) and M10 (N glucuronide, CNV3000624), the latter of which is unstable. Two additional notable circulating metabolites have been observed in humans: M14 (carboxylic acid, CNV2283325), produced by amide hydrolysis, and M16 (imine carboxylic acid, CNV2288584), which arises from oxidation of M14. In a human absorption, metabolism, and excretion study, >90% of BIIB074 and its metabolites were cleared by the urine, and the major metabolite (˜40%) excreted in urine was M13, resulting from UGT mediated metabolism of BIIB074. Thus, the PK of BIIB074 may be affected by coadministration of compounds that induce or inhibit UGTs.

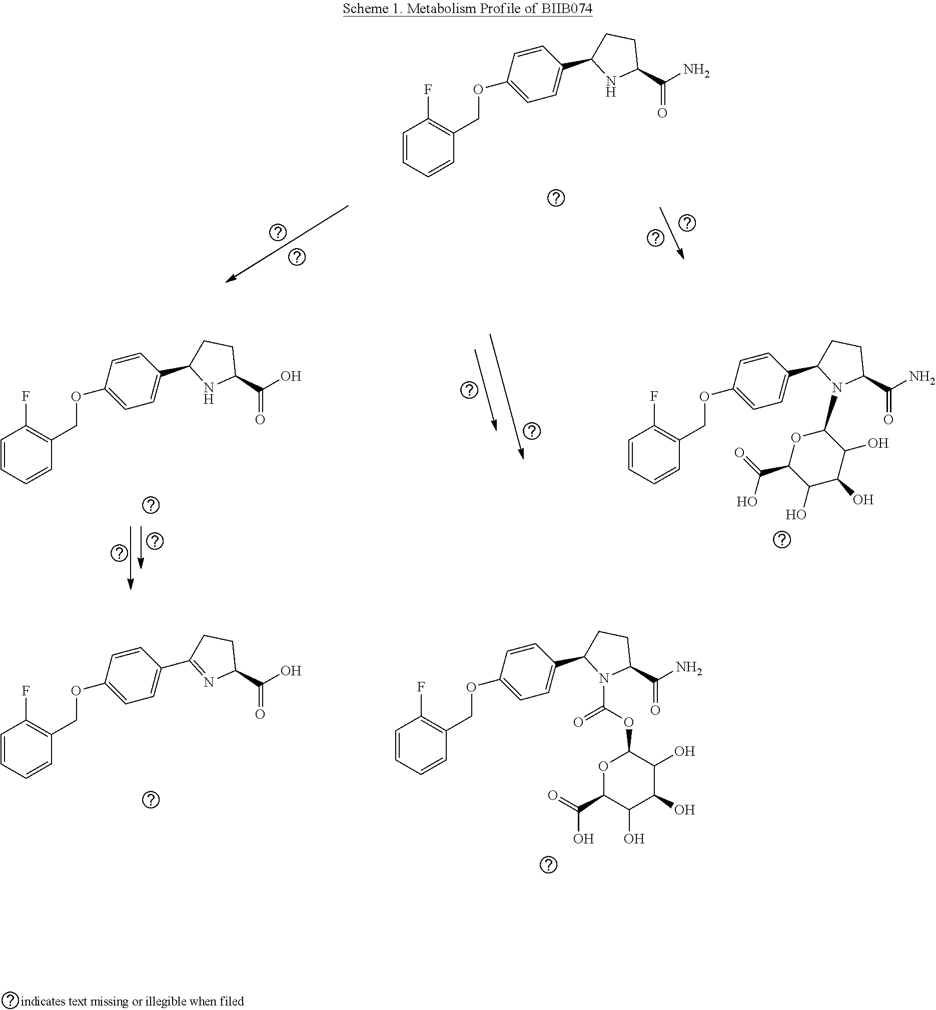 [SMILES: NC(=O)[C@@H]1CC[C@H](c2ccc(OCc3ccccc3F)cc2)N1 ; O=C(O)[C@@H]1CC[C@H](c2ccc(OCc3ccccc3F)cc2)N1 ; NC(=O)[C@@H]1CC[C@H](c2ccc(OCc3ccccc3F)cc2)N1[C@@H]1O[C@H](C(=O)O)C(O)[C@H](O)C1O ; O=C(O)[C@@H]1CCC(c2ccc(OCc3ccccc3F)cc2)=N1 ; NC(=O)[C@@H]1CC[C@H](c2ccc(OCc3ccccc3F)cc2)N1C(=O)O[C@@H]1O[C@H](C(=O)O)C(O)[C@H](O)C1O]

In clinical practice BIIB074 may be coadministered with UGT inhibitors, which could potentially increase exposure to BIIB074 by reducing the extent of BIIB074 metabolism by UGTs. Valproic acid, which has long been used as a medication to treat seizures and bipolar disorder, is a non specific inhibitor of UGTs and has been used as a probe to determine the effect of UGT inhibition on the PK of compounds that are metabolized by multiple UGTs. Herein, the potential of the UGT inhibitor valproic acid to alter the single dose PK, safety, and tolerability of BIIB074 was assessed to inform the feasibility and safety of coadministration of BIIB074 with compounds known to inhibit UGTs.

### Primary Objectives/Endpoints

To evaluate the effect of multiple doses of the uridine diphosphate glucuronosyltransferase (UGT) inhibitor valproic acid on the single dose PK of BIIB074.
   The primary endpoints that relate to this objective are: maximum observed concentration (Cₘₐₓ), area under the curve (AUC) from time 0 to infinity (AUC_{inf}), and AUC from time 0 to time of the last measurable concentration (AUCₗₐₛₜ) for BIIB074.
   Other endpoints that relate to the primary objective are the time to reach Cₘₐₓ (Tₘₐₓ), time of the last measurable concentration (Tₗₐₛₜ), t_{1/2} apparent clearance (CL/F), and apparent volume of distribution (V/F) for BIIB074.

### Secondary Objectives/Endpoints

To evaluate the safety and tolerability of BIIB074 when administered alone and when coadministered with the UGT inhibitor valproic acid.
   The endpoints that relate to this objective are the incidence of adverse events (AEs) and serious adverse events (SAEs), and changes in clinical laboratory parameters, vital signs, 12-lead electrocardiograms (ECGs), and Columbia Suicide Severity Rating Scale (C-SSRS) assessments.
To evaluate the effect of the UGT inhibitor valproic acid on the PK of the M13, M14, and M16 metabolites of BIIB074.
   The endpoints that relate to this objective are: Cₘₐₓ, AUC_{inf}, AUCₗₐₛₜ, Tₘₐₓ, Tₗₐₛₜ, t_{1/2}, and metabolite to parent ratio in AUC (MR_{AUC}) of the M13, M14, and M16 metabolites of BIIB074

### Study Design

1. A single oral dose of BIIB074 administered in the morning on Days 1 and 16 following an 8-hour fast. The Day 16 dose was coadministered with the morning dose of valproic acid.
2. Valproic acid 500 mg TID was administered on Days 8 through 22. The morning dose on Day 16 was coadministered with BIIB074 following an 8-hour fast.
3. Blood samples for BIIB074 and metabolite PK collected predose and through 168 hours postdose on Days 1 through 8 and Days 16 through 23.
4. Single blood samples collected on Days 13 through 15 prior to the morning dose of valproic acid only, in order to determine valproic acid trough levels.

### Disposition/Exposure/Populations/Narratives

30 subjects enrolled, 27 subjects completed study
   1 subject discontinued on Day 14 due to AE (vomiting) related to valproic acid
   1 subject discontinued on Day 17 (last dose of study treatment was Day 16) due to AE (vomiting) related to valproic acid
   1 subject discontinued on Day 15 due to non-compliance with the protocol (inappropriate behavior).
Safety population: 30 subjects
   2 subjects with no safety data for BIIB074 with valproic acid
PK population: 30 subjects
   2 subjects with no PK data for BIIB074 with valproic acid

### Protocol Deviations

### Major:

For all subjects, an incorrect C-SSRS form that excluded the suicidal behavior question was used on Day 8 due to staff error. With sponsor approval, the question was completed by subjects at a later date. There were no positive responses to any C-SSRS question by any subject during the study, and no AEs that were considered to be related to suicidal thoughts or tendencies.
The deviation was considered to have had no impact on the integrity of the study.

### Minor:

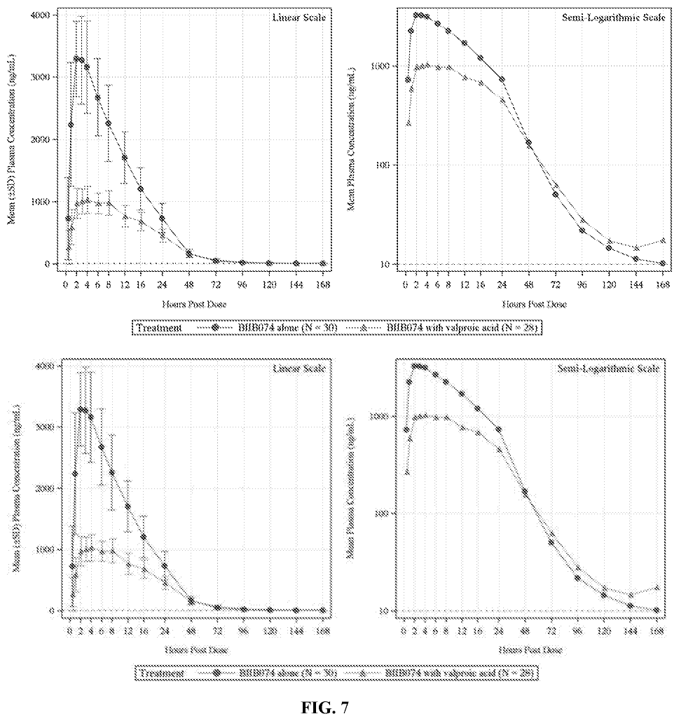

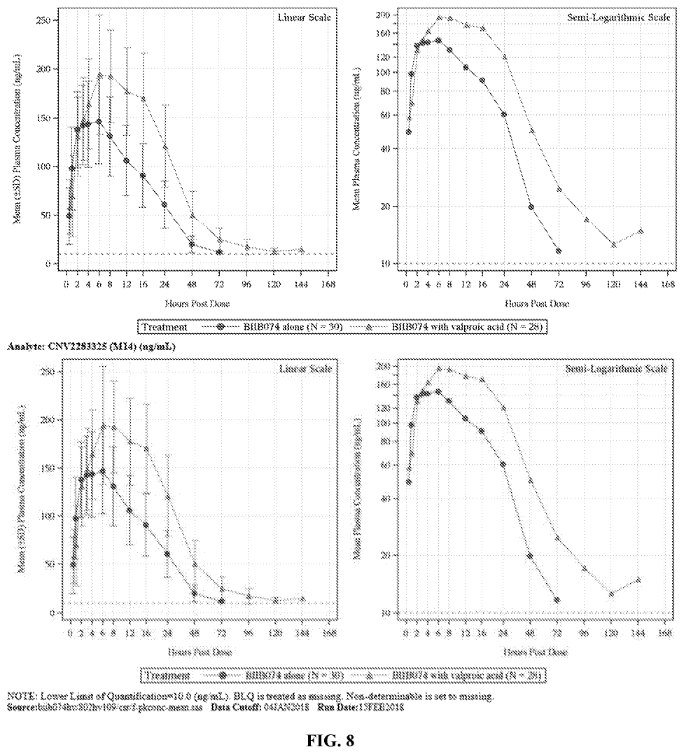

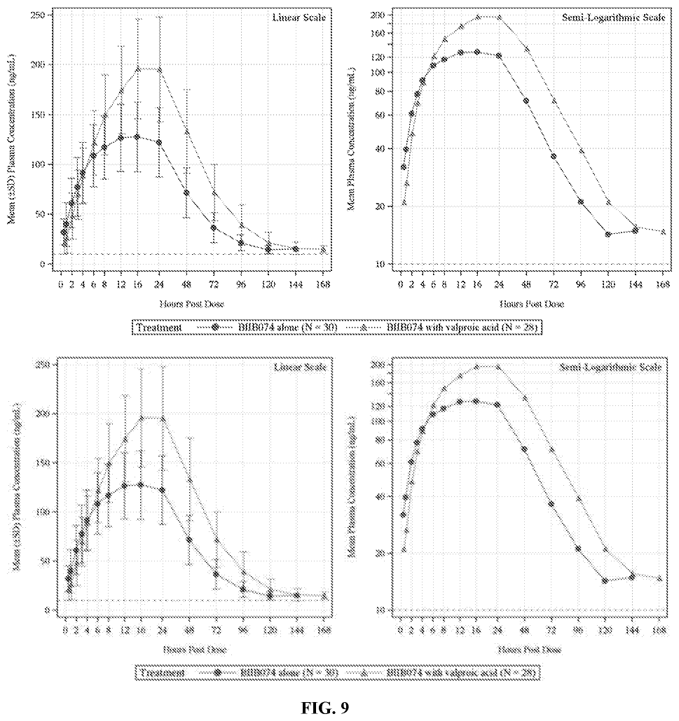

24 instances of out-of-window PK blood draws or chemistry/hematology samples
7 instances of blood samples centrifuged late
7 instances of vital signs or ECG data taken out-of-window or time/data not documented
4 instances of postdose water or posture restrictions not documented
1 instance of physical examination performed late due to PI unavailability.
These deviations were considered to have had no impact on subject safety or data integrity.
   As shown in FIG. 6, exposure of BIIB074 (AUC) increased after administration of BIIB074 with valproic acid compared to administration of BIIB074 alone. No change in Cₘₐₓ. Elimination prolonged.
   As shown in FIG. 7, exposure of the UGT-derived metabolite M13 (AUC and Cₘₐₓ) was reduced after administration of BIIB074 with valproic acid compared to BIIB074 alone.
   As shown in FIG. 8, exposure of M14 increased (AUC and Cₘₐₓ) after administration of BIIB074 with valproic acid compared to BIIB074 alone.
   As shown in FIG. 9, exposure of M16 increased (AUC and Cₘₐₓ) after administration of BIIB074 with valproic acid compared to BIIB074 alone.

TABLE 8
Summary of statistical analysis of the effect of valproic acid on the PK of BIIB074 and metabolites following treatment
with BIIB074 alone or in combination with valproic acid

| | | BIIB074 with | | | | | 90% CI of | |
|---|---|---|---|---|---|---|---|---|
| | | valproic acid | | BIIB074 alone | | Geo LS mean | Ratio | |
| Analyte | Parameter | N | Geo LS mean | N | Geo LS mean | ratio | Lower | Upper |
| BIIB074 | AUCinf (h * ng/mL) | 28 | 26929.72 | 30 | 16038.18 | 1.68 | 1.62 | 1.74 |
| | AUClast (h * ng/mL) | 28 | 26536.46 | 30 | 15742.44 | 1.69 | 1.63 | 1.74 |
| | Cmax (ng/mL) | 28 | 1264.27 | 30 | 1306.41 | 0.97 | 0.93 | 1.01 |
| CNV3000497 | AUCinf (h * ng/mL) | 28 | 28215.56 | 30 | 53961.18 | 0.52 | 0.50 | 0.55 |
| (M13) | AUClast (h * ng/mL) | 28 | 27773.23 | 30 | 53617.65 | 0.52 | 0.49 | 0.54 |
| | Cmax (ng/mL) | 28 | 1102.29 | 30 | 3440.68 | 0.32 | 0.30 | 0.34 |
| CNV2283325 | AUCinf (h * ng/mL) | 28 | 6817.47 | 29 | 3448.00 | 1.98 | 1.88 | 2.08 |
| (M14) | AUClast (h * ng/mL) | 28 | 6386.44 | 30 | 3071.67 | 2.08 | 1.95 | 2.21 |
| | Cmax (ng/mL) | 28 | 199.01 | 30 | 153.11 | 1.30 | 1.25 | 1.35 |
| CNV2288584 | AUCinf (h * ng/mL) | 28 | 12107.90 | 30 | 7147.81 | 1.69 | 1.60 | 1.80 |
| (M16) | AUClast (h * ng/mL) | 28 | 11525.02 | 30 | 6535.09 | 1.76 | 1.65 | 1.89 |
| | Cmax (ng/mL) | 28 | 200.66 | 30 | 130.14 | 1.54 | 1.44 | 1.65 |

Note:
1: Geo LS Mean = Antilog of least squares (LS) mean estimate from a mixed model analysis; Geo LS Mean Ratio = Ratio of BIIB074 with valproic acid/BIIB074 alone; 90% CI of Ratio = Exponentiated 90% Confidence Interval of the mean difference of the log-transformed data.
2: AUCinf = Area under the concentration-time curve from time zero to infinity; AUClast = Area under the concentration-time curve from time 0 to the time of last measurable concentration; Cmax = Maximum observed concentration.
3: Results obtained from a mixed effects model of natural log transformed PK parameter, including terms for treatment as a fixed effect and subject as a random effect.
4: CNV2283325 (M14) : Subject 100-113 data for PK parameter AUCinf is excluded from the summary for BIIB074 alone treatment period since extrapolated area of AUCinf > 30%.

The effects of valproic acid on BIIB074 and metabolite exposures were evaluated by calculating geometric least squares (LS) mean ratios (BIIB074 with valproic acid to BIIB074 alone).
BIIB074 exposures based on AUC_{inf} and AUCₗₐₛₜ were ˜70% higher when administered with valproic acid than when administered alone. No change in Cₘₐₓ was observed. The 90% confidence intervals (CIs) for AUCs were all above 1, and for Cₘₐₓ contained 1, indicating increased systemic exposure (AUC), but no effect for C.
The exposures of M13 based on AUCs and Cₘₐₓ were ˜50% and ˜70% lower, respectively, when BIIB074 was administered with valproic acid than when administered alone.
The exposures of M14 and M16 based on AUCs and Cₘₐₓ were higher when BIIB074 was administered with valproic acid than when administered alone.

### PK Conclusions

The plasma exposure of a single dose of BIIB074 was approximately 1.7-fold higher when administered with the UGT inhibitor valproic acid at steady state than when administered alone. The elimination phase of BIIB074 was prolonged in the presence of valproic acid, as reflected by an increased fin value.
The plasma exposure of M13, the UGT glucuronide metabolite of BIIB074, was approximately 50% lower based on AUC and approximately 70% lower based on Cₘₐₓ when a single dose of BIIB074 was administered with valproic acid at steady state than when administered alone. The MR_{AUC} for M13 was lower when a single dose of BIIB074 was administered with valproic acid at steady state (MR_{AUC}=0.6) than when administered alone (MR_{AUC}=2.0), consistent with reduced UGT-mediated metabolism of BIIB074.

TABLE 9
Overall Summary of Adverse Effects

| | BIIB074 alone | Valproic acid alone | BIIB074 with valproic acid | Overall |
|---|---|---|---|---|
| | (N = 30) | (N = 30) | (N = 28) | (N = 30) |
| | n (%) | n (%) | n (%) | n (%) |
| Number of subjects with | 2 (6.7) | 6 (20.0) | 7 (25.0) | 10 (33.3) |
| Any event | | | | |
| Severity (a) | | | | |
| Mild | 2 (6.7) | 6 (20.0) | 7 (25.0) | 10 (33.3) |
| Moderate | 0 | 0 | 0 | 0 |
| Severe | 0 | 0 | 0 | 0 |
| Related event (b) | 0 | 0 | 0 | 0 |
| Serious event | 0 | 0 | 0 | 0 |
| Related serious event (b) | 0 | 0 | 0 | 0 |
| Events leading to drug | 0 | 0 | 0 | 0 |
| withdrawal | | | | |
| Events leading to study | 0 | 1 (3.3) | 1 (3.6) | 2 (6.7) |
| withdrawal | | | | |
| Fatal event | 0 | 0 | 0 | 0 |

NOTE
1: A subject can appear in more than one category.
(a) Each subject counted once at maximum severity.
(b) Related to BIIB074 as assessed by the investigator
Drug withdrawal refers to BIIB074 withdrawal AEs with a start date/time after the subject's Clinic Discharge or ETV date/time through the Safety Follow-up Telephone Call are only counted in the overall column.

TABLE 10
Analysis of Adverse Effects

| | BIIB074 alone | Valproic acid alone | BIIB074 with valproic acid | Overall |
|---|---|---|---|---|
| | (N = 30) | (N = 30) | (N = 3228) | (N = 3230) |
| | n (%) | n (%) | n (%) | n (%) |
| Number of subjects with any | 2 (6.7) | 6 (20.0) | 7 (25.0) | 10 (33.3) |
| event | | | | |
| Gastrointestinal disorders | 1 (3.3) | 4 (13.3) | 7 (25.0) | 9 (30.0) |
| Nausea | 0 | 4 (13.3) | 7 (25.0) | 9 (30.0) |
| Vomiting | 0 | 2 (6.7) | 2 (7.1) | 3 (10.0) |
| Diarrhoea | 0 | 1 (3.3) | 1 (3.6) | 2 (6.7) |
| Dyspepsia | 0 | 2 (6.7) | 0 | 2 (6.7) |
| Abdominal pain | 0 | 0 | 1 (3.6) | 1 (3.3) |
| Faeces soft | 0 | 1 (3.3) | 0 | 1 (3.3) |
| Glossodynia | 1 (3.3) | 0 | 0 | 1 (3.3) |
| Nervous system disorders | 1 (3.3) | 2 (6.7) | 2 (7.1) | 3 (10.0) |
| Headache | 1 (3.3) | 1 (3.3) | 2 (7.1) | 3 (10.0) |
| Dizziness | 0 | 1 (3.3) | 1 (3.6) | 1 (3.3) |
| General disorders and | 0 | 1 (3.3) | 1 (3.6) | 2 (6.7) |
| administration | | | | |
| site conditions | | | | |
| Asthenia | 0 | 0 | 1 (3.6) | 1 (3.3) |
| Non-cardiac chest pain | 0 | 1 (3.3) | 0 | 1 (3.3) |
| Vascular disorders | 0 | 0 | 2 (7.1) | 2 (6.7) |
| Pallor | 0 | 0 | 2 (7.1) | 2 (6.7) |
| Cardiac disorders | 0 | 1 (3.3) | 1 (3.6) | 1 (3.3) |
| Skin and subcutaneous tissue | 0 | 1 (3.3) | 0 | 1 (3.3) |
| disorders | | | | |
| Pruritus | 0 | 1 (3.3) | 0 | 1 (3.3) |
| Palpitations | 0 | 1 (3.3) | 1 (3.6) | 1 (3.3) |

NOTE
1: Within each treatment period and overall a subject was counted only once within each system organ class and preferred term (MedDRA version 20.0).
2: System organ class and preferred term are presented in decreasing frequency of the table's rightmost column.
3: AEs with a start date/time after the subject's Clinic Discharge or ETV date/time through the Safety Follow-up Telephone Call are only counted in the overall column.

### Table 10. Analysis of AEs

The most frequently reported TEAEs overall by preferred term were nausea (9 [30.0%] subjects), headache (3 [10.0%] subjects), vomiting (3 [10.0%] subjects), diarrhoea (2 [6.7%] subjects), dyspepsia (2 [6.7%] subjects), and pallor (2 [6.7%] subjects). All other TEAEs were reported in only a single subject.
All TEAEs of nausea, vomiting, diarrhoea, and dyspepsia were reported in subjects in the valproic acid alone or BIIB074 with valproic acid treatment groups and were considered related to valproic acid.
No TEAEs were considered related to BIIB074
   Vital Signs, ECGs, Physical Examinations, C-SSRS, and Safety Conclusion Vitals signs: Subjects in all treatment groups had sporadic clinically relevant vital signs values, but none of these were considered to be clinically significant or reported as a TEAE.
   12-Lead ECGs: No subjects had an increase in QTcF from baseline>30 msec or an absolute QTcF>450 msec for males or >460 msec for females. Subjects in all treatment groups had sporadic out-of-range ECG values or shifts to abnormal findings, but none of these were considered to be clinically significant or reported as a TEAE.
   Physical Examinations: No abnormal findings in postdose physical examinations were reported as ILAEs.
   C-SSRS: No suicide-related events were reported based on C-SSRS assessments.

### Safety Conclusion:

BIIB074 was safe and well tolerated in this study when administered alone and when administered with valproic acid.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a disease or condition mediated by modulation of Nav1.7, comprising administering (5R)-5-(4-{[(2-fluorophenyl)methyl]oxy}phenyl)-L-prolinamide, or a pharmaceutically acceptable salt thereof, to a subject not receiving treatment with a UGT inhibitor.

2. The method of claim 1, wherein the disease or condition is associated with a defect or dysfunction of Nav1.7.

3. The method of claim 1, wherein the UGT inhibitor is selected from canagliflozin, dapagliflozin, mefenamic acid, probenecid, diclofenac, quinidine, fluconazole, and valproic acid.

4. The method of claim 1, wherein the method further comprises
a) determining whether the subject is receiving treatment with a UGT inhibitor and
b) if the subject is receiving treatment with a UGT inhibitor, discontinuing treatment with the UGT inhibitor prior to commencing treatment with (5R)-5-(4-{[(2-fluorophenyl)methyl]oxy}phenyl)-L-prolinamide, or a pharmaceutically acceptable salt thereof, or
c) if the subject is not receiving treatment with a UGT inhibitor, instructing the subject not to commence treatment with a UGT inhibitor while receiving treatment with (5R)-5-(4-{[(2-fluorophenyl)methyl]oxy}phenyl)-L-prolinamide, or a pharmaceutically acceptable salt thereof.

5. (canceled)

6. The method of claim 1, wherein discontinuing treatment with the UGT inhibitor comprises discontinuing treatment with the UGT inhibitor at least three weeks before commencing treatment with (5R)-5-(4-{[(2-fluorophenyl)methyl]oxy}phenyl)-L-prolinamide, or a pharmaceutically acceptable salt thereof.

7. The method of claim 1, wherein administering (5R)-5-(4-{[(2-fluorophenyl)methyl]oxy}phenyl)-L-prolinamide, or a pharmaceutically acceptable salt thereof, comprises administering (5R)-5-(4-{[(2-fluorophenyl)methyl]oxy}phenyl)-L-prolinamide, or a pharmaceutically acceptable salt thereof, one time per day (OID), two times per day (BID), or three times per day (TID).

8. (canceled)

9. (canceled)

10. The method of claim 1, wherein administering (5R)-5-(4-{[(2-fluorophenyl)methyl]oxy}phenyl)-L-prolinamide, or a pharmaceutically acceptable salt thereof, comprises administering (5R)-5-(4-{[(2-fluorophenyl)methyl]oxy}phenyl)-L-prolinamide, or a pharmaceutically acceptable salt thereof, at a dosage of about 150 mg to about 400 mg.

11-17. (canceled)

18. The method of claim 1, wherein administering (5R)-5-(4-{[(2-fluorophenyl)methyl]oxy}phenyl)-L-prolinamide, or a pharmaceutically acceptable salt thereof, comprises administering (5R)-5-(4-{[(2-fluorophenyl)methyl]oxy}phenyl)-L-prolinamide, or a pharmaceutically acceptable salt thereof, at a dosage of about 300 mg to about 400 mg two times per day (BID).

19. The method of claim 18, wherein the dosage is about 300 mg BID.

20. (canceled)

21. The method of claim 19, wherein the method further comprises administering (5R)-5-(4-{[(2-fluorophenyl)methyl]oxy}phenyl)-L-prolinamide, or a pharmaceutically acceptable salt thereof, at a dosage of about 400 mg BID for an initial period of time prior to administering (5R)-5-(4-{[(2-fluorophenyl)methyl]oxy}phenyl)-L-prolinamide, or a pharmaceutically acceptable salt thereof, at a dosage of about 300 mg BID.

22. (canceled)

23. The method of claim 18, wherein the dosage is about 400 mg BID.

24-26. (canceled)

27. The method of claim 1, wherein the disease or condition is pain.

28. The method of claim 27, wherein the pain is neuropathic pain.

29. The method of claim 28, wherein the neuropathic pain is selected from diabetic neuropathy; sciatica; non-specific lower back pain; painful lumbosacral radiculopathy; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions.

30. The method of claim 28, wherein the neuropathic pain is selected from trigeminal neuralgia, painful lumbosacral radiculopathy, erythromelalgia, and small fibre neuropathy.

31. The method of claim 28, wherein the neuropathic pain is trigeminal neuralgia.

32. The method of claim 31, comprising administering (5R)-5-(4-{[(2-fluorophenyl)methyl]oxy}phenyl)-L-prolinamide, or a pharmaceutically acceptable salt thereof, to the subject at a dosage of about 250 mg three times per day (TID).

33. The method of claim 28, wherein the neuropathic pain is painful lumbosacral radiculopathy.

34. The method of claim 33, comprising administering (5R)-5-(4-{[(2-fluorophenyl)methyl]oxy}phenyl)-L-prolinamide, or a pharmaceutically acceptable salt thereof, to the subject at a dosage of about 200 mg two times per day (BID).

35. (canceled)

36. (canceled)

37. A method of treating a disease or condition mediated by modulation of Nav1.7, comprising administering (5R)-5-(4-{[(2-fluorophenyl)methyl]oxy}phenyl)-L-prolinamide, or a pharmaceutically acceptable salt thereof, to a subject receiving treatment with a UGT inhibitor.

38-62. (canceled)
